SEQUENCE LISTING (4 sequences)

SEQ ID NO: 1 - RNA, 21 nt, synthetic construct
  source 1..21: mol_type = other RNA
cagccccttattgttatacga

SEQ ID NO: 2 - RNA, 21 nt, synthetic construct
  source 1..21: mol_type = other RNA
tcgtataacaataaggggctg

SEQ ID NO: 3 - RNA, 21 nt, synthetic construct
  source 1..21: mol_type = other RNA
  modified_base 1..8: mod_base = OTHER; note = 2'-O-methyl nucleotide
  modified_base 9..11: mod_base = OTHER; note = 2'-deoxy-2'-fluoro nucleotide
  modified_base 21: mod_base = OTHER; note = deoxynucleotide
  modified_base 1..2: mod_base = OTHER; note = phophorothioate bond linking nucleotides at positions 1 and 2
  modified_base 20..21: mod_base = OTHER; note = phosphorothioate bond linking nucleotides at positions 20 and 21
  modified_base 20..21: mod_base = OTHER; note = nucleotides at positions 20 and 21 are 3'- 3' linked
  modified_base 1: mod_base = OTHER; note = nucleotide at position 1 is attached to R1 by a phosphorothioate bond, wherein R1 is an n-acetylgalactosamine glycopeptide
  modified_base 12..20: mod_base = OTHER; note = 2'-O-methyl nucleotide
cagccccttattgttatacga

SEQ ID NO: 4 - RNA, 21 nt, synthetic construct
  source 1..21: mol_type = other RNA
  modified_base 1: mod_base = OTHER; note = 2'-O-methyl nucleotide
  modified_base 3: mod_base = OTHER; note = 2'-O-methyl nucleotide
  modified_base 5: mod_base = OTHER; note = 2'-O-methyl nucleotide
  modified_base 7..11: mod_base = OTHER; note = 2'-O-methyl nucleotide
  modified_base 13: mod_base = OTHER; note = 2'-O-methyl nucleotide
  modified_base 15: mod_base = OTHER; note = 2'-O-methyl nucleotide
  modified_base 17: mod_base = OTHER; note = 2'-O-methyl nucleotide
  modified_base 19: mod_base = OTHER; note = 2'-O-methyl nucleotide
  modified_base 21: mod_base = OTHER; note = 2'-O-methyl nucleotide
  modified_base 2: mod_base = OTHER; note = 2'-deoxy-2'-fluoro nucleotide
  modified_base 4: mod_base = OTHER; note = 2'-deoxy-2'-fluoro nucleotide
  modified_base 6: mod_base = OTHER; note = 2'-deoxy-2'-fluoro nucleotide
  modified_base 12: mod_base = OTHER; note = 2'-deoxy-2'-fluoro nucleotide
  modified_base 14: mod_base = OTHER; note = 2'-deoxy-2'-fluoro nucleotide
  modified_base 16: mod_base = OTHER; note = 2'-deoxy-2'-fluoro nucleotide
  modified_base 18: mod_base = OTHER; note = 2'-deoxy-2'-fluoro nucleotide
  modified_base 20: mod_base = OTHER; note = 2'-deoxy-2'-fluoro nucleotide
  modified_base 1..2: mod_base = OTHER; note = the nucleotides at positions 1 and 2 are linked by a phosphorothioate bond
  modified_base 2..3: mod_base = OTHER; note = the nucleotides at positions 2 and 3 are linked by a phosphorothioate bond
  modified_base 19..20: mod_base = OTHER; note = the nucleotides at positions 19 and 20 are linked by a phosphorothioate bond
  modified_base 20..21: mod_base = OTHER; note = the nucleotides at positions 20 and 21 are linked by a phosphorothioate bond
tcgtataacaataaggggctg

(19) United States
(12) Patent Application Publication
(10) Pub. No.: US 2025/0171765 A1
(43) Pub. Date: May 29, 2025

(54) METHODS OF PREPARING HIGH CONCENTRATION LIQUID DRUG SUBSTANCES

(71) Applicant: AMGEN INC., Thousand Oaks, CA (US)

(72) Inventors: Namita SAWANT, Burlington, MA (US); Sai Prasanth CHAMARTHY, Oak Park, CA (US); Arnold MCAULEY, Moorpark, CA (US); Swagat SAHU, Simi Valley, CA (US)

(73) Assignee: AMGEN INC., Thousand Oaks, CA (US)

(21) Appl. No.: 18/840,615

(22) PCT Filed: Feb. 24, 2023
(86) PCT No.: PCT/US2023/063247

§ 371 Date: Aug. 22, 2024

Related U.S. Application Data
(60) Provisional application No. 63/313,840, filed on Feb. 25, 2022.

(51) Int. Cl. C12N 15/10; A61K 9/00; A61K 9/08; A61K 31/7105

(52) U.S. Cl. CPC C12N 15/101; A61K 9/0019; A61K 9/08; A61K 31/7105; C12N 15/1017; C12N 2310/14

(57) ABSTRACT

Provided herein are methods of preparing a high concentration, liquid composition comprising an oligonucleotide compound. In exemplary embodiments, the method comprises exchanging by diafiltration an oligonucleotide compound in a starting solution into a diafiltration (DF) solution to obtain an intermediate solution, wherein the concentration of the oligonucleotide compound in the starting solution is 140 mg/mL or less and the DF solution comprises one or more salts, and concentrating by ultrafiltration the oligonucleotide compound in the intermediate solution to obtain a high concentration, liquid composition, wherein the concentration of the oligonucleotide compound in the high concentration, liquid composition is greater than about 150 mg/mL.

### CROSS REFERENCE TO RELATED APPLICATIONS

The benefit under 35 U.S.C. § 119(e) of U.S. Provisional Application No. 63/313,840, filed Feb. 25, 2022, is hereby claimed, and the disclosure thereof is hereby incorporated by reference herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 20.5 KB file named “A-2897-WO01-SEC_Seq_Listing.xml”; created on Feb. 20, 2023.

### BACKGROUND

Oligonucleotide drug products are on the rise, as the number of oligonucleotides approved for commercial therapeutic use has more than doubled in the past five years, and the number of oligonucleotide programs in development have tripled in the past ten years (Muslehiddinoglu et al., Nucleic Acid Therapeutics 30(4): 189-197 (2020)). All oligonucleotide drug products currently on the market are formulated for parenteral administration, including intravitreal, intravenous, intrathecal, intramuscular or subcutaneous administration. For many, the oligonucleotide drug substance (DS) used for drug product (DP) manufacturing is a lyophilized powder. Lyophilized drug substances are easy to transport and store, are at low risk for microbial growth, and exhibit stability for greater than three years under refrigerated and frozen conditions. However, lyophilization is an energy-intensive, time-consuming, and costly process. From the standpoints of batch output and cycle time, lyophilization can add up to five days to the manufacturing process thereby increasing inefficiency to the overall process. Also, as lyophilized drug products require reconstitution with formulation buffer, dissolution, compounding and dilution, the overall complexity of the manufacturing process is increased when it includes lyophilization (Muslehiddinoglu et al., 2020, supra).

A flow diagram of a traditional manufacturing process of an oligonucleotide compound from a powder (lyophilized) DS is shown in FIG. 1A. The oligonucleotide DS is manufactured as a lyophilized powder that can be stored at 2-8° C. or −20° C. During DP manufacturing process, the DS powder is thawed to controlled room temperature. After thawing, the powder is weighed and transferred to the compounding vessel for further reconstitution with the formulation buffer to the desired concentration, followed by thorough mixing to manufacture the final bulk drug product ready for filtration and fill operations.

Instead of a lyophilized powder, aqueous solutions of oligonucleotide DS have been proposed as a more efficient manufacture-to-administration pathway (Muslehiddinoglu et al., 2020, supra). A liquid DS solution would allow for an easier DP manufacturing process flow involving thaw of the frozen DS solution followed by mixing, filtration and fill processes without the need for powder handling and reconstitution. A flow diagram of an exemplary manufacturing process of an oligonucleotide compound from a liquid drug substance is shown in FIG. 1B. In addition to simplifying the overall manufacturing process, the cost of manufacturing would decrease.

Solution DS and DP are not without challenges, as stability concerns and microbial contamination risks may arise. Transportation of solutions in large masses can also lead to complicated supply chain issues, especially if the solutions are stored frozen. Additionally, the manufacture of solution DS or DP typically involves ultrafiltration/diafiltration (UF/DF). Challenges associated with UF/DF could include, low fluxes, long process times, mechanical recovery yield and losses, operator-intensive intervention or handling, low mass transfer rates, energy inefficiencies, and hydraulic pressure limits on concentration equipment. While active pharmaceutical ingredient (API) concentrations of 40-150 mg/mL may be achieved in solution with UF/DF (Muslehiddinoglu et al., 2020, supra), the maximum concentration of oligonucleotide compounds achievable with UF/DF is limited by the interactions of the oligonucleotides with the membrane resulting in membrane fouling. Higher target API concentrations would likely to lead to membrane fouling which slows the transfer of water and smaller ions through the membrane, which in turn would lengthen the time to reach the desired concentration if not prevent achievement of the target API concentration altogether. Increasing the molecular weight cut-off (MWCO) of the membrane used in UF/DF could facilitate the process but could also lead to reduced recovery of the oligonucleotide. Such challenges could limit achieving high concentration oligonucleotide DP manufactured using liquid DS.

Thus, a new process for preparing an oligonucleotide compound, e.g., an oligonucleotide DS, at a high concentration as a liquid composition (e.g., a solution) is needed. The process would, for instance, lead to the preparation of liquid compositions comprising the oligonucleotide compound at a concentration of greater than about 150 mg/mL. Ideally, such a process would avoid the need for lyophilization and thus avoid reconstitution and dissolution of a DS powder.

### SUMMARY

Presented herein are data supporting the feasibility of a method for preparing a high concentration, liquid composition comprising an oligonucleotide compound. As detailed herein, the method achieves preparation of liquid compositions comprising the oligonucleotide compound at concentrations as high as about 150 mg/mL, and, in various instances, the method achieves even higher concentrations of the oligonucleotide compound. As described herein, the method achieves, for instance, a liquid composition comprising an oligonucleotide compound at a concentration greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL, or greater than about 230 mg/mL.

Without being bound to a particular theory, the achievement of such high concentration, liquid compositions are, at least in part, attributed to the solution in which the oligonucleotide compound exists during ultrafiltration, which, in exemplary aspects, relates to the diafiltration (DF) solution used in the method. Without being bound by theory, the solution in which the oligonucleotide compound exists during ultrafiltration, or the DF solution used for diafiltration, comprises one or more salts which interact with the oligonucleotide compound in a way which stabilizes the oligonucleotide compound and/or increases its hydrodynamic diameter. Without being bound to a particular theory, the salt(s) of the solution, e.g., DF solution, interact(s) with the oxygen atoms and/or sulfur atoms of the oligonucleotide compound via, e.g., hydrogen bonds, metal coordination, electrostatic interactions. Without being bound to a particular theory, the more interactions made between the oligonucleotide compound and the salt(s), the higher the concentration of the oligonucleotide compound may be achieved. Such interactions are associated with the absence of or a minimization of fouling of the membrane used in the method.

Accordingly, provided herein are methods for preparing a high concentration, liquid composition comprising an oligonucleotide compound, wherein the liquid composition comprises the oligonucleotide compound at a concentration greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL. In exemplary embodiments, the method of preparing the high concentration, liquid composition comprises: (a) preparing an oligonucleotide compound in a first solution comprising one more salts, wherein the concentration of the oligonucleotide compound in the first solution is 140 mg/mL or less, and the total salt concentration of the first solution is about 25 mM to about 800 mM, and (b) concentrating by ultrafiltration the first solution to obtain a high concentration, liquid composition comprising the oligonucleotide compound at a concentration of about 150 mg/mL or greater. In various aspects, the first solution is prepared by diafiltration with a DF solution. In various aspects, the first solution is the same as the DF solution except that the first solution comprises the oligonucleotide compound and the DF solution does not comprise the oligonucleotide compound. In various instances, the diafiltration achieves an exchange of a starting solution comprising the oligonucleotide compound at about 140 mg/mL or less into a DF solution comprising one or more salts and having a total salt concentration of 25 mM to about 800 mM. Accordingly, in exemplary embodiments, the method of preparing the high concentration liquid composition comprises: (a) exchanging by diafiltration an oligonucleotide compound in a starting solution into a DF solution to obtain an intermediate solution, wherein the concentration of the oligonucleotide compound in the starting solution is 140 mg/mL or less, and the DF solution comprises one or more salts, and (b) concentrating by ultrafiltration the oligonucleotide compound in the intermediate solution to obtain a high concentration, liquid composition, wherein the concentration of the oligonucleotide compound in the high concentration, liquid composition is about 150 mg/ml or greater. In exemplary instances, the total salt concentration of the DF solution is about 25 mM to about 800 mM. Optionally, the total salt concentration of the DF solution is about 25 mM to about 500 mM. In exemplary instances, the method comprises continuous diafiltration and/or ultrafiltration by tangential flow filtration. In various aspects, the method employs a polyethersulfone (PES) membrane or a stabilized cellulose membrane for the diafiltration and/or ultrafiltration. Optionally, the membrane has a molecular weight cut-off (MWCO) of less than 10 kDa, and in exemplary instances, the MWCO is about 5 kDa or about 3 kDa.

Also provided herein are methods of concentrating by ultrafiltration a first solution comprising a low concentration of an oligonucleotide compound to obtain a second solution comprising a high concentration of the oligonucleotide compound, wherein the oligonucleotide compound concentration of the second solution is greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL. In exemplary aspects, the oligonucleotide compound concentration of the first solution is less than about 140 mg/mL, less than about 130 mg/mL, less than about 120 mg/mL, less than about 110 mg/mL, less than about 100 mg/mL, less than about 90 mg/mL, less than about 80 mg/mL, less than about 70 mg/mL, less than about 60 mg/mL, or less than about 50 mg/mL. In various aspects, the second solution is a retentate obtained upon ultrafiltration. In various aspects, the retentate is achieved after 1 or 2 hours of ultrafiltration. In exemplary instances, the oligonucleotide compound is double stranded. In exemplary instances, the total salt concentration of the first solution is about 25 mM to about 800 mM. Optionally, the total salt concentration of the first solution is about 25 mM to about 500 mM or about 25 mM to about 250 mM. In various aspects, the first solution comprises only one salt. Optionally, the one salt is an inorganic salt, such as, any of the inorganic salts described herein. In various aspects, the first solution does not comprise any acetate. In exemplary instances, the method of concentrating is carried out after diafiltration with a DF solution. In various aspects, the first solution is the same as the DF solution except that the first solution comprises the oligonucleotide compound and the DF solution does not comprise the oligonucleotide compound. In various instances, the diafiltration is carried out with a DF solution comprising one or more salts and the total salt concentration of the DF solution is about 25 mM to about 800 mM. Optionally, the total salt concentration of the DF solution is about 25 mM to about 500 mM or about 25 mM to about 250 mM. In exemplary aspects, the method of concentrating is carried out before diafiltration with a DF solution. In various instances, the oligonucleotide compound is a double-stranded oligonucleotide compound, optionally, an siRNA.

Further provided herein is a method of preparing a solution API comprising an oligonucleotide compound API, wherein the oligonucleotide compound API is present in the solution at a concentration greater than 150 mg/mL. In various aspects, the oligonucleotide compound API is double-stranded, optionally, an siRNA. In exemplary embodiments, the method of preparing a solution API comprises a presently disclosed method of concentrating by ultrafiltration a first solution comprising a low concentration of an oligonucleotide compound to obtain a second solution comprising a high concentration of the oligonucleotide compound. In various instances, the method of preparing a solution API comprises (i) synthesizing the oligonucleotide compound, or a strand thereof, by, e.g., solid phase synthesis, (ii) carrying out one or more of a chromatography, a diafiltration, and an annealing, and (iii) concentrating by ultrafiltration in accordance with a presently disclosed method of concentrating by ultrafiltration a first solution comprising a low concentration of an oligonucleotide compound to obtain a second solution comprising a high concentration of the oligonucleotide compound. In various aspects, the oligonucleotide compound concentration of the second solution is greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL, and the oligonucleotide compound concentration of the first solution is less than about 140 mg/mL, less than about 130 mg/mL, less than about 120 mg/mL, less than about 110 mg/mL, less than about 100 mg/mL, less than about 90 mg/mL, less than about 80 mg/mL, less than about 70 mg/mL, less than about 60 mg/mL, or less than about 50 mg/mL. In various aspects, the second solution is a retentate obtained upon ultrafiltration. In various aspects, the retentate is achieved after 1 or 2 hours of ultrafiltration. In exemplary aspects of the method of preparing a solution API comprising an oligonucleotide compound API, the oligonucleotide API is not lyophilized at any point during the method. Advantageously, the method of preparing the solution API is a method which lacks any lyophilization. Accordingly, the method of preparing the solution API of the present disclosure is free of a lyophilization and provides a more time- and cost-efficient way of preparing an oligonucleotide compound API. In various instances, the solution API prepared by the presently disclosed method is directly used for preparing a solution DP comprising the oligonucleotide compound API. Optionally, the solution API is sterile filtered and filled into vials or pre-filled syringes or autoinjectors. Accordingly, a method of producing a DP, e.g., a solution DP, comprising a solution API comprising an oligonucleotide compound API is provided. In various aspects, the method of producing the DP lacks any lyophilization. In various aspects of the method of producing the DP, lyophilization of the oligonucleotide compound API is not carried out at any time during the method.

In exemplary aspects, the method of the present disclosure leads to a high concentration, liquid composition, wherein the concentration of the oligonucleotide compound in the high concentration, liquid composition is about 150 mg/ml or greater. In exemplary aspects, the high concentration, liquid composition obtained by the presently disclosed method comprises greater than 80% of the oligonucleotide compound of the starting solution. In exemplary instances, the method achieves a 70% or an 80% recovery of the oligonucleotide compound of the starting solution. In exemplary aspects, membrane flux is substantially maintained during the method, e.g., during the diafiltration and/or ultrafiltration. In various instances, membrane flux is substantially maintained for at least 6 hours, at least 8 hours, or greater than 9 hours. In exemplary aspects, little to no membrane fouling occurs during the method, e.g., during the diafiltration and/or ultrafiltration. In exemplary instances, a high concentration, liquid composition comprising the oligonucleotide compound at a concentration of about 150 mg/mL or greater is obtained, and the membrane flux decreases by not more than 50% of the steady state membrane flux, which optionally is maintained for at least 4 hours or more.

The present disclosure also provides a high concentration, liquid composition prepared by the method of the present disclosure. In various instances, the high concentration, liquid composition comprises the oligonucleotide compound at a concentration of about 150 mg/ml or greater, e.g., about 175 mg/mL, about 180 mg/mL, about 185 mg/mL, about 190 mg/mL, about 195 mg/mL, about 200 mg/mL, about 205 mg/mL, about 210 mg/mL, about 215 mg/mL, about 220 mg/mL, about 225 mg/mL, about 230 mg/mL, about 235 mg/mL, about 240 mg/mL, about 245 mg/mL, about 250 mg/mL, or greater. A frozen preparation made by storing the presently disclosed liquid composition at a temperature below 0 degrees C. is additionally provided. The frozen preparation in exemplary aspects is not a lyophilized or freeze-dried preparation.

The present disclosure further provides methods of manufacturing a medicament comprising an oligonucleotide compound. In exemplary embodiments, the method comprises carrying out the method of the present disclosure to obtain a high concentration, liquid composition comprising greater than about 150 mg/mL of the oligonucleotide compound, formulating the high concentration, liquid composition with a pharmaceutically acceptable excipient, and filling the formulated high concentration, liquid composition into a container.

Methods of treating a subject with a disease are furthermore provided. In exemplary embodiments, the method comprises administering to the subject a liquid composition of the present disclosure, e.g., a liquid DP, in an amount effective to treat the disease in the subject. Optionally, the medicament is administered by injection or infusion.

Use of the high concentration, liquid composition of the present disclosure in the manufacture of a medicament for treating a disease in a subject is moreover provided.

### BRIEF DESCRIPTION OF THE DRAWINGS

FIG. 1A is a flow diagram of a drug product manufacturing process with a powder drug substance. FIG. 1B is a flow diagram of a drug product manufacturing process with a bulk liquid drug substance.

FIG. 2 is a diagram of an exemplary method for preparing a high concentration, liquid composition comprising an oligonucleotide compound. The direction of movement of solutions is shown with solid black arrows.

FIG. 3 depicts the structure of a model oligonucleotide compound schematically. The top strand listed in the 5′ to 3′ direction is the sense strand (SEQ ID NO: 3) and the bottom strand listed in the 3′ to 5′ direction is the antisense strand (SEQ ID NO: 4). Black circles represent nucleotides with a 2′-O-methyl modification, white circles represent nucleotides with a 2′-deoxy-2′-fluoro (“2′-fluoro”) modification, and the gray circle represents a deoxyadenosine nucleotide linked to the adjacent nucleotide via a 3′-3′ linkage (i.e. inverted). Gray lines connecting the circles represent phosphodiester linkages, whereas the black lines connecting the circles represent phosphorothioate linkages. A trivalent GalNAc moiety having the depicted structure is represented by R1 and is covalently attached to the 5′ end of the sense strand by a phosphorothioate linkage.

FIG. 4 is a chromatogram showing the membrane flux (top) and pressure (bottom) across a 5 kDa Hydrosart stabilized cellulose membrane in Ambr Crossflow set up using two different DF solutions: (i) 1×PBS and (ii) a buffer solution comprising 20 mM phosphate buffer with 40 mM NaCl.

FIG. 5 is a graph of the unfolding temperature of an oligonucleotide compound placed in a DF solution comprising various salt concentrations.

FIG. 6 is a graph of the hydrodynamic diameter of an oligonucleotide compound placed in a DF solution comprising various salt concentrations.

FIG. 7 is a simplified schematic of an exemplary manufacturing process for a double-stranded oligonucleotide compound comprising a sense strand and antisense strand. Each strand is separately synthesized and subsequently purified, buffer exchanged, and/or concentrated via chromatography and/or UF/DF prior to annealing the sense and antisense strands. After annealing, the double-stranded oligonucleotide compound is buffer exchanged and concentrated via UF/DF to obtain a high concentration solution of drug substance (double-stranded oligonucleotide compound) wherein the concentration of the double-stranded oligonucleotide compound is greater than 150 mg/mL. The high concentration solution of drug substance is used to prepare DP and is sterile filtered and filled into containers. Optionally, the high concentration solution of drug substance is exchanged into a formulation buffer before sterile filtration and filling. Optionally, the DP is stored after sterile filtration and filling. Lyophilization is not carried out at any point during this process.

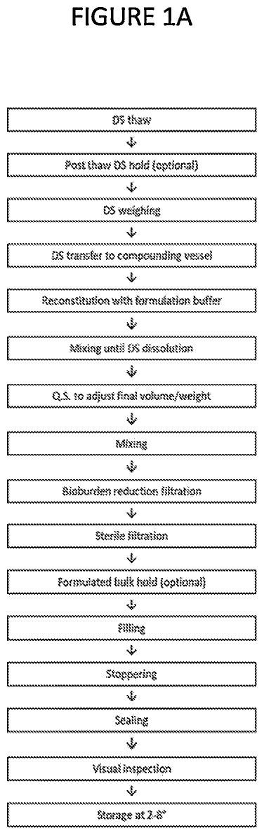

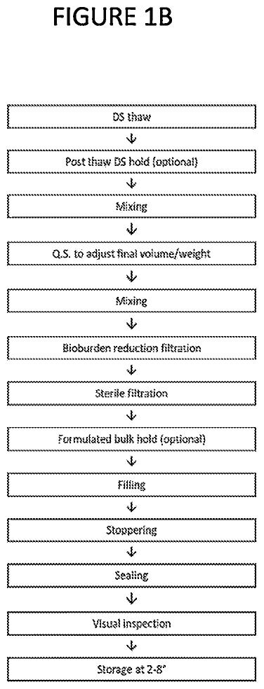

### DETAILED DESCRIPTION

Provided herein are methods for preparing a high concentration, liquid composition comprising an oligonucleotide compound. In exemplary embodiments, the method entails preparation of a liquid composition comprising an oligonucleotide compound at a high concentration, e.g., greater than or about 150 mg/mL, and the method comprises: (a) preparing a first solution comprising an oligonucleotide compound and one or more salts, wherein the concentration of the oligonucleotide compound in the first solution is 140 mg/mL or less and the total salt concentration of the first solution is about 25 mM to about 800 mM, and (b) concentrating by ultrafiltration the first solution to obtain a high concentration, liquid composition comprising the oligonucleotide compound at a concentration of greater than about 150 mg/mL. In various aspects, the first solution is prepared by diafiltration wherein the oligonucleotide compound in a starting solution is exchanged into a first solution or a diafiltration (DF) solution to obtain an intermediate solution, which is then concentrated by ultrafiltration to obtain a high concentration, liquid composition. In various aspects, the first solution is the same as the DF solution except that the first solution comprises the oligonucleotide compound and the DF solution does not comprise the oligonucleotide compound. The first solution may be the same as any DF solution described herein but also includes the oligonucleotide compound at a low concentration (e.g., less than about 140 mg/mL). In exemplary embodiments, the method comprises: (a) exchanging by diafiltration an oligonucleotide compound in a starting solution into a diafiltration (DF) solution to obtain an intermediate solution, wherein the concentration of the oligonucleotide compound in the starting solution is 140 mg/mL or less and the DF solution comprises one or more salts, and (b) concentrating by ultrafiltration the oligonucleotide compound in the intermediate solution to obtain a high concentration, liquid composition, wherein the concentration of the oligonucleotide compound in the high concentration, liquid composition is about 150 mg/mL or greater. In exemplary aspects, the high concentration, liquid composition obtained by the presently disclosed methods comprises the oligonucleotide compound at a concentration of about 150 mg/mL or greater, e.g., about 160 mg/mL or greater, about 170 mg/mL or greater, about 180 mg/mL or greater, about 190 mg/mL or greater, about 200 mg/mL or greater, about 210 mg/mL or greater, about 220 mg/mL or greater, about 230 mg/mL or greater, about 240 mg/mL or greater, about 250 mg/ml or greater, about 260 mg/mL or greater, about 270 mg/mL or greater, about 280 mg/mL or greater, about 290 mg/ml or greater, about 300 mg/mL or greater. In exemplary instances, the presently disclosed methods achieve a high recovery of the oligonucleotide compound present in the starting solution. In various instances, the method achieves at least a 70% or at least at 80% recovery of the oligonucleotide compound of the starting solution. In various aspects, the method achieves at least an 85% (e.g., at least a 90%, at least a 95%, at least a 98%) recovery of the oligonucleotide compound present in the starting solution. In exemplary aspects, the high concentration liquid composition obtained by the presently disclosed methods comprises greater than 80% of the oligonucleotide compound present in the starting solution. In various instances, the high concentration liquid composition obtained by the presently disclosed methods comprises greater than 85% (e.g., greater than 90%, greater than 95%, greater than 98%) of the oligonucleotide compound present in the starting solution. Without being bound by theory, the methods of the present disclosure achieve such high levels of recovery of the oligonucleotide compound, and such high concentrations of the oligonucleotide compound in the obtained liquid composition, because the diafiltration and/or ultrafiltration occur(s) without substantial membrane fouling. In various instances, membrane flux is substantially maintained during the diafiltration and/or ultrafiltration. In various aspects, the membrane flux is substantially maintained for at least 6 hours, at least 8 hours, at least 10 hours, at least 12 hours, or longer. In various aspects, the membrane flux reaches a steady state level and decreases by not more than 50% (optionally, not more than 60%, not more than 70%, not more than 80%, not more than 90%) during the diafiltration and/or ultrafiltration. Given that the membrane flux is substantially maintained without substantial membrane fouling, the methods of the present disclosure are advantageously more efficient. In various aspects, the maintained membrane flux and lack of substantial membrane fouling leads to the attainment of the high concentration liquid composition in a shorter period of time, compared to a different method which is slowed by substantial membrane fouling. Thus, the methods of the present disclosure are advantageously marked by shorter process times to achieve the high concentration liquid composition. In various aspects, the process time to achieve the high concentration liquid composition is less than 36 hours, less than 32 hours, less than 30 hours, less than 28 hours, less than 26 hours, less than 24 hours, less than 22 hours, less than 20 hours, less than 18 hours, less than 16 hours, less than 14 hours, less than 12 hours, or less than 10 hours. Without being bound to any particular theory, the method of the present disclosure provides an efficient way of buffer exchanging a starting solution comprising the oligonucleotide compound into a DF solution and concentrating the oligonucleotide compound in the DF solution, without any substantial membrane fouling, without significant loss of the oligonucleotide compound (e.g., with good recovery of the oligonucleotide compound), and/or within a relatively short period of time (e.g., about 10 hours or less). The presently disclosed methods advantageously avoid the need for the energy-intensive and time-consuming lyophilization of the oligonucleotide compound, and thus, is more efficient.

In exemplary embodiments, the method comprises exchanging by diafiltration an oligonucleotide compound in a starting solution into a diafiltration (DF) solution to obtain an intermediate solution. In various aspects, the oligonucleotide compound starts in a starting solution comprising a first component or set of components and the method comprises transferring the oligonucleotide compound from the starting solution into a diafiltration solution which is different in composition from the starting solution and comprises a different component or set of components. This exchange from the starting solution to the diafiltration solution in various aspects is known in the art as a “buffer exchange” and is achieved by diafiltration. The term “diafiltration” or “DF” refers to a process for exchanging a large molecule product from one solution or buffer to another. DF is often employed for buffer exchange. In various aspects, diafiltration is carried out to exchange the starting solution for the DF solution (so the oligonucleotide compound starts in the starting solution and is effectively transferred into the DF solution) to obtain an intermediate solution. In various aspects, the intermediate solution comprises the oligonucleotide compound and the DF solution. Optionally, the intermediate solution comprises at least 70% of the amount of the oligonucleotide compound present in the starting solution. In various aspects, the intermediate solution comprises at least 75%, at least 80%, at least 85%, or at least 90% of the amount of the oligonucleotide compound present in the starting solution. In various instances, the diafiltration achieves at least a 70% or at least 80% recovery of the oligonucleotide compound of the starting solution. In various aspects, the method achieves at least an 85% (e.g., at least a 90%, at least a 95%, at least a 98%) recovery of the oligonucleotide compound present in the starting solution.

In exemplary embodiments, the method comprises concentrating the oligonucleotide compound in the intermediate solution. In exemplary embodiments, the method comprises increasing the concentration of the oligonucleotide compound of the intermediate solution by concentrating and the concentrating is achieved by ultrafiltration. The term “ultrafiltration” or “UF” refers to a process for separating large molecule compounds from small molecule components (e.g., water, cations, anions) using a membrane for the purposes of desalting or concentrating solutions comprising the large molecule compounds.

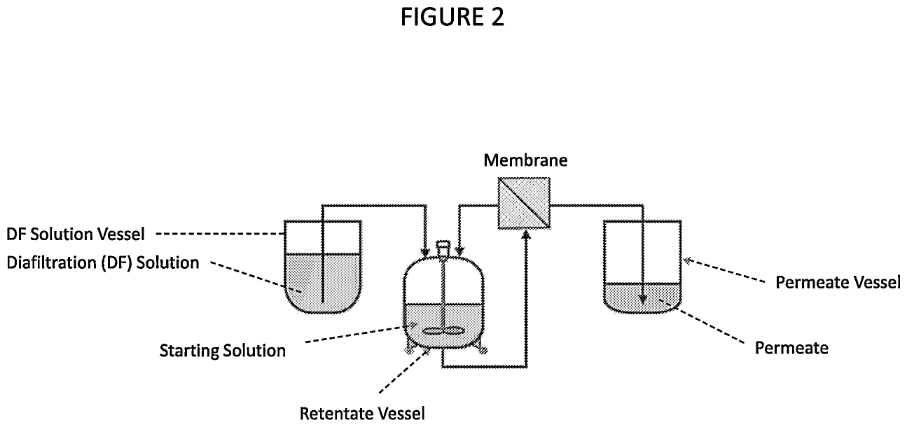

UF and DF are well described in the art. See, e.g., Millipore Mechanical Brief: Protein Concentration and Diafiltration by Tangential Flow Filtration, available at chrome-extension://efaidnbmnnnibpcajpcglclefindmkaj/viewer.html?pdfurl=http%3A%2F%2Fwolfson.huji.ac.il%2Fpurification%2FPDF%2Fdialysis%2FMILLIPORE_TFF.pdf&clen=2842762&chunk=true See, e.g., Cheryan, M. Ultrafiltration Handbook, Technomic Publishing Co., Inc., Pennsylvania (1986); Koros et al., Pure & Appl Chem 68:1479-1489; Ng et al., Separation Science 2:499-502 (1976); Tkacik and Michaels, Bio/Technol 9:941-946 (1991); van Reis et al., Biotech Bioeng 56:71-82 (1997); van Reis et al., J Membrane Sci 130:123-140 (1997); van Reis and Zydney, Protein Ultrafiltration In M. C. Flikinger, S. W. Drew (eds.), Encyclopedia of Bioprocess Technology, John Wiley and Sons, Inc., New York (1999); Zeman and Zydney, Microfiltration and Ultrafiltration: Principles and Applications, Marcel Dekker, New York (1996); U.S. Patent Application Publication Nos. 2006/0051347 and 2014/0370003; Maruthamuthu et al., Trends in Biotechnology, 38(10): 1169-1186 (2020); Kovács, Z. (2016). Continuous Diafiltration: Cocurrent and Countercurrent Modes. In: Drioli, E., Giorno, L. (eds) Encyclopedia of Membranes. Springer, Berlin, Heidelberg. https://doi.org/10.1007/978-3-662-44324-8_638; and Schwartz, L., Introduction to Tangential Flow Filtration for Laboratory and Process Development Applications, the contents of each are incorporated herein by reference. In various aspects, DF and UF are carried out in an exemplary UF/DF system shown in FIG. 2. In various aspects, the UF/DF system comprises a pump (not shown in FIG. 2) which causes movement of the solutions in the directions of the arrows. In exemplary instances, as DF solution is added to a starting solution in the retentate vessel to create a mixture, the pump causes movement of the solution in the retentate vessel to move through the membrane to obtain a retentate, which comprises the oligonucleotide compound, and a permeate. The pump causes movement of the retentate to the retentate vessel and the permeate to the permeate vessel. The movement of the solutions continues in this manner until the volume of the DF solution added to the retentate vessel is essentially the same as the volume of permeate collected in the permeate vessel. The volume of the DF solution added to the retentate vessel in various instances is about 3 times the volume of the starting solution. Optionally, the volume of the DF solution is about 4 times or 5 times the volume of the starting solution. In various aspects, the volume of the starting solution is referred to as a “diavolume” and optionally, the volume of the DF solution added to the retentate vessel is 3, 4, or 5 diavolumes. In various instances, the volume of the DF solution added to the retentate vessel is 6, 7, 8, 9, or 10 diavolumes. Generally, the higher the number of diavolumes the greater percent exchange is accomplished via diafiltration. In exemplary instances, the method comprises continuous diafiltration and/or ultrafiltration by tangential flow. In various aspects, a membrane is employed to carry out the diafiltration and ultrafiltration. In various instances, a membrane comprising cellulose acetate, polyvinylidene fluoride (PVDF), or polyethersulfone (PES) is used. Optionally, the membrane used for the diafiltration and/or ultrafiltration is a polyethersulfone (PES) membrane or a stabilized cellulose membrane. In various aspects, the membrane has a molecular weight cut-off (MWCO) of less than 10 kDa, and in exemplary instances, the MWCO is about 5 kDa. In various aspects, the membrane is a cellulose membrane having a 3 kDa MWCO. Accordingly, in exemplary aspects, the method comprises (a) adding a diafiltration (DF) solution comprising one or more salts to a starting solution comprising the oligonucleotide compound at a concentration of 140 mg/mL or less to create a mixture; (b) passing the mixture through a membrane to obtain a retentate and a permeate, wherein the retentate comprises the oligonucleotide compound, and (c) reducing the volume of the retentate to obtain a final retentate, wherein the concentration of the oligonucleotide compound in the final retentate is at least about 150 mg/mL. In various instances, prior to (a), the starting solution is contained in a retentate vessel, and the method comprises adding the DF solution to the retentate vessel to create the mixture. Optionally, the DF solution is added from a DF solution vessel to the retentate vessel to create the mixture. In exemplary embodiments, a total volume of the DF solution is added over at least 30 minutes, at least 60 minutes, at least 2 hours, at least 4 hours, at least 6 hours or at least 8 hours. In various aspects, the method comprises (b1) passing the retentate to the retentate vessel and the permeate is passed to a permeate vessel, wherein the retentate in the retentate vessel mixes with DF solution and starting solution to create an admixture. In various instances, the method comprises (b2) passing the admixture through the membrane to obtain a retentate and a permeate. Optionally, the method comprises repeating (b1) and (b2) at least one or two times or repeating (b1) and (b2) at least two or more times, until the volume of the permeate in the permeate vessel is substantially the same as the volume of the DF solution added to the retentate vessel. In various aspects, two or more of (a), (b), (b1), and (b2) occur simultaneously. In exemplary aspects, the DF solution vessel, retentate vessel, permeate vessel, and membrane are part of an ultrafiltration/diafiltration (UF/DF) system. The UF/DF system further comprises a pump in certain aspects, and the pump moves (i) DF solution from the DF solution vessel to the retentate vessel, (ii) the solution from the retentate vessel through the membrane, (iii) the retentate from the membrane to the retentate vessel, (iv) the permeate to the permeate vessel, or (v) a combination thereof. Optionally, the DF solution is moved to the retentate vessel at a rate that is similar to, or the same as, the rate at which the permeate is collected. In various aspects, the method comprises comprising reducing the volume of a solution in the retentate vessel, when the volume of the permeate collected in the permeate vessel is essentially the same as the volume of the DF solution moved from the DF solution vessel to the retentate vessel. In various instances, the volume of a solution in the retentate vessel is reduced by ceasing movement of DF solution from the DF solution vessel to the retentate vessel. Optionally, the membrane is part of a membrane cassette. The method of the present disclosure comprises reducing the volume of the retentate in the retentate vessel to obtain a final retentate. In various aspects, the volume is reduced via ultrafiltration, wherein the volume of the retentate is reduced by moving the retentate to/through the membrane without DF solution being fed into the retentate vessel while collecting permeate in the permeate vessel. The volume of the permeate collected in the permeate vessel is equivalent to the volume of the DF solution fed into the retentate vessel and the concentration of the oligonucleotide in the final retentate is 150 mg/ml or higher. In exemplary aspects, the volume of the retentate is reduce by at least 50% relative to the volume of the starting solution. In various instances, the method comprises reducing the volume of the retentate to obtain a final retentate, wherein the concentration of the oligonucleotide compound in the final retentate is greater than or about 150 mg/mL. In various aspects, the final retentate comprises the oligonucleotide compound at a concentration of at least 155 mg/mL, at least 160 mg/mL, at least 165 mg/mL, at least 170 mg/mL, at least 175 mg/mL, at least 180 mg/mL, at least 185 mg/mL, at least 190 mg/mL, at least 195 mg/mL, or at least 200 mg/mL. In various instances, the final retentate comprises the oligonucleotide compound at a concentration of at least 210 mg/mL, at least 220 mg/mL, at least 230 mg/mL, of higher. In various instances, the final retentate comprises the oligonucleotide compound at a concentration of at least 250 mg/mL, at least 275 mg/ml, at least 300 mg/mL, at least 325 mg/mL, at least 350 mg/mL, at least 375 mg/mL, at least 400 mg/mL, or higher. Optionally, the concentration of the oligonucleotide compound of the final retentate is at least 2-times higher than the concentration of the oligonucleotide compound in the starting solution. In various aspects, the concentration of the oligonucleotide compound of the final retentate is at least 3-times or at least 4-times higher than the concentration of the oligonucleotide compound in the starting solution. In exemplary instances, the concentration of the oligonucleotide compound of the final retentate is at least 5-times higher than the concentration of the oligonucleotide compound in the starting solution.

The presently disclosed methods for preparing a high concentration liquid of an oligonucleotide compound utilize a starting solution and a DF solution. In various aspects, the starting solution comprises the oligonucleotide compound at a lower concentration than the concentration of the high concentration liquid composition. In various aspects, the concentration of the oligonucleotide compound in the starting solution is less than 150 mg/mL, less than 140 mg/mL, less than 130 mg/mL, less than 120 mg/mL, less than 110 mg/mL, less than 100 mg/mL, less than 90 mg/mL, less than 80 mg/mL, less than 70 mg/mL, less than 60 mg/mL, or less than 50 mg/ml (e.g., less than 45 mg/mL, less than 40 mg/mL, less than 35 mg/mL, less than 30 mg/mL, less than 25 mg/mL, less than 20 mg/mL, less than 15 mg/mL, less than 10 mg/mL, less than 5 mg/mL). In various aspects, the concentration of the oligonucleotide compound in the starting solution is about 30 mg/mL to about 140 mg/mL, about 30 mg/mL to about 130 mg/mL, about 30 mg/mL to about 120 mg/mL, about 30 mg/mL to about 110 mg/mL, about 30 mg/mL to about 100 mg/mL, about 30 mg/mL to about 90 mg/mL, about 30 mg/mL to about 80 mg/mL, about 30 mg/mL to about 70 mg/mL, about 30 mg/mL to about 60 mg/mL, about 30 mg/mL to about 50 mg/mL, about 30 mg/mL to about 40 mg/mL, about 40 mg/mL to about 140 mg/mL, about 50 mg/mL to about 140 mg/mL, about 60 mg/mL to about 140 mg/mL, about 70 mg/mL to about 140 mg/mL, about 80 mg/mL to about 140 mg/mL, about 90 mg/mL to about 140 mg/mL, about 100 mg/mL to about 140 mg/mL, about 110 mg/mL to about 140 mg/mL, about 120 mg/mL to about 140 mg/mL, or about 130 mg/mL to about 140 mg/mL. In various aspects, the concentration of the oligonucleotide compound in the starting solution is about 30 mg/mL to about 100 mg/mL. In various aspects, the concentration of the oligonucleotide compound in the starting solution is about 50 mg/mL to about 140 mg/mL, about 50 mg/mL to about 130 mg/mL, about 50 mg/mL to about 120 mg/mL, about 50 mg/mL to about 110 mg/mL, about 50 mg/mL to about 100 mg/mL, about 50 mg/mL to about 90 mg/mL, about 50 mg/mL to about 80 mg/mL, about 50 mg/mL to about 70 mg/mL, about 50 mg/mL to about 60 mg/mL, about 60 mg/mL to about 140 mg/mL, about 70 mg/mL to about 140 mg/mL, about 80 mg/mL to about 140 mg/mL, about 90 mg/mL to about 140 mg/mL, about 100 mg/mL to about 140 mg/mL, about 110 mg/mL to about 140 mg/mL, about 120 mg/mL to about 140 mg/mL, or about 130 mg/mL to about 140 mg/mL. In various aspects, the concentration of the oligonucleotide compound in the starting solution is about 50 mg/ml to about 100 mg/ml. In exemplary instances, the starting solution comprises water or a buffer. In exemplary instances, the starting solution is free of buffer. Advantageously, the method of the present disclosure is not limited to the components of the starting solution.

The DF solution, which may also be referred to as a replacement solution, or in some instances, a replacement buffer or diafiltration buffer, comprises at least one salt. In various aspects, the total salt concentration of the DF solution is about 25 mM to about 800 mM or about 50 mM to about 800 mM. In exemplary aspects, the total salt concentration of the DF solution is about 50 mM to about 750 mM, about 50 mM to about 700 mM, about 50 mM to about 650 mM, about 50 mM to about 600 mM, about 50 mM to about 550 mM, about 50 mM to about 500 mM, about 50 mM to about 450 mM, about 50 mM to about 400 mM, about 50 mM to about 350 mM, about 50 mM to about 300 mM, about 50 mM to about 250 mM, about 50 mM to about 200 mM, about 50 mM to about 150 mM, about 50 mM to about 100 mM, about 50 mM to about 75 mM, about 75 mM to about 800 mM, about 100 mM to about 800 mM, about 150 mM to about 800 mM, about 200 mM to about 800 mM, about 250 mM to about 800 mM, about 300 mM to about 800 mM, about 350 mM to about 800 mM, about 400 mM to about 800 mM, about 450 mM to about 800 mM, about 500 mM to about 800 mM, about 550 mM to about 800 mM, about 600 mM to about 800 mM, about 650 mM to about 800 mM, about 700 mM to about 800 mM, or about 750 mM to about 800 mM. In exemplary aspects, the total salt concentration is less than 500 mM. Optionally, the total salt concentration is about 25 mM to about 500 mM, about 25 mM to about 400 mM, about 25 mM to about 300 mM, about 25 mM to about 250 mM, about 25 mM to about 200 mM, or about 25 mM to about 150 mM. In various aspects, the DF solution comprises, consists essentially of, or consists of an inorganic salt comprising a monovalent cation and the total salt concentration of the DF solution is about 25 mM to about 500 mM, about 25 mM to about 400 mM, about 25 mM to about 300 mM, about 25 mM to about 250 mM, about 25 mM to about 200 mM, or about 25 mM to about 150 mM. In various aspects, the total salt concentration is about 75 mM to about 500 mM, about 75 mM to about 400 mM, about 75 mM to about 300 mM, about 75 mM to about 250 mM, about 75 mM to about 200 mM, or about 75 mM to about 150 mM. In various aspects, the DF solution comprises, consists essentially of, or consists of an inorganic salt comprising a monovalent cation and the total salt concentration of the DF solution is about 75 mM to about 500 mM, about 75 mM to about 400 mM, about 75 mM to about 300 mM, about 75 mM to about 250 mM, about 75 mM to about 200 mM, or about 75 mM to about 150 mM. In various instances, the total salt concentration of the DF solution is about 125 mM to about 500 mM. Optionally, the total salt concentration of the DF solution is about 150 mM to about 500 mM, about 175 mM to about 500 mM, about 200 mM to about 500 mM, about 225 mM to about 500 mM, about 250 mM to about 500 mM, about 275 mM to about 500 mM, about 300 mM to about 500 mM, about 325 mM to about 500 mM, about 350 mM to about 500 mM, about 375 mM to about 500 mM, about 400 mM to about 500 mM, about 425 mM to about 500 mM, about 450 mM to about 500 mM, about 475 mM to about 500 mM, about 125 mM to about 475 mM, about 125 mM to about 450 mM, about 125 mM to about 425 mM, about 125 mM to about 400 mM, about 125 mM to about 375 mM, about 125 mM to about 350 mM, about 125 mM to about 325 mM, about 125 mM to about 300 mM, about 125 mM to about 275 mM, about 125 mM to about 250 mM, about 125 mM to about 225 mM, about 125 mM to about 200 mM, about 125 mM to about 175 mM, or about 125 mM to about 150 mM. In exemplary aspects, the total salt concentration of the DF solution is about 140 mM to about 300 mM. Optionally, the total salt concentration of the DF solution is about 140 mM to about 280 mM, about 140 mM to about 260 mM, about 140 mM to about 240 mM, about 140 mM to about 220 mM, about 140 mM to about 200 mM, about 140 mM to about 180 mM, about 140 mM to about 160 mM, about 160 mM to about 300 mM, about 180 mM to about 300 mM, about 200 mM to about 300 mM, about 220 mM to about 300 mM, about 240 mM to about 300 mM, about 260 mM to about 300 mM, or about 280 mM to about 300 mM. In various instances, the total salt concentration of the DF solution is about 50 mM to about 900 mM, about 50 mM to about 875 mM, about 50 mM to about 850 mM, about 50 mM to about 800 mM, about 50 mM to about 775 mM, about 50 mM to about 750 mM, or about 50 mM to about 700 mM, e.g., about 60 mM to about 700 mM, about 70 mM to about 700 mM, about 80 mM to about 700 mM, about 90 mM to about 700 mM, about 100 mM to about 700 mM, about 110 mM to about 700 mM, about 120 mM to about 700 mM, about 130 mM to about 700 mM, about 140 mM to about 700 mM, about 150 mM to about 700 mM, about 160 mM to about 700 mM, about 170 mM to about 700 mM, about 180 mM to about 700 mM, about 190 mM to about 700 mM, about 200 mM to about 700 mM, about 210 mM to about 700 mM, about 220 mM to about 700 mM, about 230 mM to about 700 mM, about 240 mM to about 700 mM, about 250 mM to about 700 mM, about 260 mM to about 700 mM, about 270 mM to about 700 mM, about 280 mM to about 700 mM, about 290 mM to about 700 mM, about 300 mM to about 700 mM, about 310 mM to about 700 mM, about 320 mM to about 700 mM, about 330 mM to about 700 mM, about 340 mM to about 700 mM, about 350 mM to about 700 mM, about 360 mM to about 700 mM, about 370 mM to about 700 mM, about 380 mM to about 700 mM, about 390 mM to about 700 mM, about 400 mM to about 700 mM. In various instances, the total salt concentration of the DF solution is about 100 mM to about 900 mM, about 200 mM to about 900 mM, about 300 mM to about 900, about 400 mM to about 900 mM, about 500 mM to about 900 mM, about 600 mM to about 900 mM, about 700 mM to about 900 mM or about 800 mM to about 900 mM. In various instances, the total salt concentration of the DF solution is about 100 mM to about 700 mM, optionally, about 100 mM to about 650 mM, about 100 mM to about 600 mM, about 100 mM to about 550 mM, about 100 mM to about 500 mM, about 100 mM to about 450 mM, about 100 mM to about 400 mM, about 100 mM to about 350 mM, about 100 mM to about 300 mM, about 100 mM to about 250 mM, about 100 mM to about 200 mM, about 100 mM to about 150 mM, about 150 mM to about 700 mM, about 200 mM to about 700 mM, about 250 mM to about 700 mM, about 300 mM to about 700 mM, about 350 mM to about 700 mM, about 400 mM to about 700 mM, about 450 mM to about 700 mM, about 500 mM to about 700 mM, about 550 mM to about 700 mM, about 600 mM to about 700 mM, or about 650 mM to about 700 mM. The total salt concentration of the DF solution is about 100 mM to about 600 mM, in various aspects. Optionally, the total salt concentration of the DF solution is about 125 mM to about 300 mM, optionally, about 125 mM to about 200 mM or about 125 mM to about 150 mM. In exemplary aspects, the total salt concentration of the DF solution is about 130 mM to about 140 mM. In various instances, the DF solution is substantially free of potassium. Optionally, the DF solution comprises a molar ratio of salt, e.g., primary salt, to potassium of about 100 to 1.

In exemplary aspects, the first solution or DF solution, comprises at least one salt and the total salt concentration of the DF solution is about 25 mM to about 800 mM or about 50 mM to about 800 mM. Optionally, the total salt concentration of the DF solution is less than 500 mM, e.g., about 25 mM to about 500 mM. In exemplary instances, the DF solution comprises, consists essentially of, or consists of an inorganic salt. As used herein, the term “inorganic salt” refers to any inorganic compound with ionic bonds that do not contain a carbon-hydrogen bond. Inorganic salts, include, but are not limited to, transition metal salts, alkali metal salts, post-transition metal salts, metalloid salts, alkaline earth metal salts, lanthanide salts, ammonium salts, halogen inorganic salts, reactive non-metal salts, and xenon salts. Suitable inorganic salts include any of those described at the catalog described at https://www.thermofisher.com/search/browse/category/us/en/80013663/inorganic-salts, and are not toxic or destabilizing to the oligonucleotide compound. In exemplary aspects, the inorganic salt is an alkali metal salt, an alkaline earth metal salt or an ammonium salt. In various aspects, the alkali metal salt is any inorganic salt the includes an element in group 1a of the periodic table. Alkali metal salts include, e.g., sodium chloride, lithium chloride, potassium chloride, sodium bromide, potassium hydrogen phosphate, sodium hydroxide, potassium carbonate, cesium hydroxide, lithium hydroxide, potassium hydroxide, sodium dihydrogen phosphate, sodium acetate, sodium fluoride, potassium titanium oxide, sodium carbonate, lithium amide, and the like. In various aspects, the alkaline earth metal salt is any inorganic salt includes an element in group 2 of the periodic table. Alkaline earth metal salts include, for instance, calcium chloride, magnesium chloride, magnesium oxide, calcium carbonate, strontium fluoride, barium sulfate, calcium chromate, calcium DL-glycerate dihydrate, barium maganate, magnesium silicate, calcium hydride, calcium nitrate tetrahydrate, strontium chloride hexahydrate, magnesium carbonate hydroxide pentahydrate, strontium bromide, calcium sulfate dihydrate, magnesium niobium oxide, methylmagnesium chloride, magnesium thiosulfate, magnesium perchlorate, calcium perchlorate, barium acetate, and the like. In exemplary instances, the ammonium salt is any inorganic salt that has an ammonium cation. Ammonium salts include, for example, ammonium bicarbonate, ammonium persulfate, ammonium carbonate, ammonium hydroxide, ammonium formate, ammonium cobalt (II) phosphate, ammonium copper (II) sulfate, ammonium sulfamate, ammonium oxalate, ammonium perchlorate, ammonium nitrate, ammonium bromide, ammonium dichromate, ammonium dihydrogen phosphate, and the like. In various aspects, the inorganic salt comprises a monovalent cation. Optionally, the inorganic salt comprises an alkali metal, optionally, sodium, potassium, or lithium. In various aspects, the inorganic salt comprises a halogen counterion, optionally chloride or bromide. In various instances, the inorganic salt is sodium chloride, sodium bromide, potassium chloride, or lithium chloride. In some aspects, the monovalent cation is an ammonium cation, optionally, wherein the inorganic salt is ammonium chloride. In alternative aspects, the inorganic salt comprises a divalent cation. Optionally, the divalent cation is an alkaline earth metal, optionally, magnesium or calcium. In exemplary instances, the inorganic salt is magnesium chloride or calcium chloride. In exemplary instances, the DF solution comprises, consists essentially of, or consists of an organic salt. As used herein, the term “organic salt” refers to a salt containing an organic ion which contains at least one carbon-hydrogen bond and typically comprise only covalent bonds. Organic salts generally comprise only one or more of carbon, hydrogen, oxygen, sulfur, nitrogen, and phosphorus atoms. The organic salt in exemplary instances comprises a quaternary ammonium cation. Optionally, the organic salt is choline chloride or benzyltrimethylammonium chloride. In various aspects, the total salt concentration of the DF solution is about 25 mM to about 500 mM, about 25 mM to about 250 mM, or about 25 mM to about 150 mM and the DF solution comprises, consists essentially of, or consists of an inorganic salt comprising a divalent cation, optionally, calcium chloride or magnesium chloride. In various instances, the total salt concentration of the DF solution is about 75 mM to about 300 mM, about 75 mM to about 250 mM, or about 75 mM to about 200 mM and the DF solution comprises, consists essentially of, or consists of an inorganic salt comprising a monovalent cation. In exemplary instances, the DF solution comprises about 75 mM to about 300 mM sodium chloride, sodium bromide, lithium chloride, potassium chloride, or ammonium chloride. In various instances the total salt concentration of the DF solution is about 100 mM to about 300 mM and the DF solution comprises, consists essentially of, or consists of an organic salt, e.g., CCl or BTMACl.

In various aspects, the total salt concentration of the DF solution depends on the net number of negative charges of the oligonucleotide compound. For example, for an oligonucleotide comprising 41 net negative charges, the total salt concentration is about 50 mM to about 800 mM, or any of the total salt concentrations of the DF solution described herein. In various instances, the total salt concentration of the DF solution is expressed as concentration (e.g., mM) per negative charge of the oligonucleotide compound. Thus, in various instances, when the oligonucleotide compound comprises 41 net negative charges, the concentration per net negative charge may be multiplied by 41. For example, in various aspects, the total salt concentration of the DF solution per negative charge is about 3.0 mM to 18.0 mM. Optionally, the total salt concentration of the DF solution per negative charge is about 3.0 mM to 17.0 mM, about 3.0 mM to 16.0 mM, about 3.0 mM to 15.0 mM, about 3.0 mM to 14.0 mM, about 3.0 mM to 13.0 mM, about 3.0 mM to 12.0 mM, about 3.0 mM to 11.0 mM, about 3.0 mM to 10.0 mM, about 3.0 mM to 9.0 mM, about 3.0 mM to 8.0 mM, about 3.0 mM to 7.0 mM, about 3.0 mM to 6.0 mM, about 3.0 mM to 5.0 mM, about 3.0 mM to 4.0 mM, about 4.0 mM to 18.0 mM, about 5.0 mM to 18.0 mM, about 6.0 mM to 18.0 mM, about 7.0 mM to 18.0 mM, about 8.0 mM to 18.0 mM, about 9.0 mM to 18.0 mM, about 10.0 mM to 18.0 mM, about 11.0 mM to 18.0 mM, about 12.0 mM to 18.0 mM, about 13.0 mM to 18.0 mM, about 14.0 mM to 18.0 mM, about 15.0 mM to 18.0 mM, about 16.0 mM to 18.0 mM, or about 17.0 mM to 18.0 mM. In various aspects, the total salt concentration of the DF solution per negative charge of the oligonucleotide compound is at least about 3.5 mM, at least about 3.6, at least about 3.7, at least about 3.8, at least about 3.9, or at least about 4.0. In exemplary aspects, the DF solution comprises one or two salts. In various instances, the DF solution comprises more than two salts, optionally, 3, 4, 5, 6, or more salts.

In various instances, the DF solution comprises more than one salt, and one salt, hereinafter referred to as the “primary salt” is present in the DF solution at a substantially higher concentration compared to the other salt(s) of the DF solution. Alternatively, the DF solution comprises only one salt which may be considered as the “primary salt”. In various aspects, the DF solution comprises a primary salt and at least one other salt. In various aspects, the concentration of the primary salt is at least 2-times greater, at least 3-times greater, at least 4-times greater, or at least 5-times greater, than the concentration of another salt present in the DF solution. In various instances, the concentration of the primary salt is about 10-times greater than the concentration of another salt present in the DF solution. In various aspects, the concentration of the primary salt is at least 2-times greater, at least 3-times greater, at least 4-times greater, or at least 5-times greater, than the concentration of all other salts present in the DF solution. In various instances, the concentration of the primary salt is about 10-times greater than the concentration of all other salts present in the DF solution. In various instances, the DF solution comprises a buffer. In various aspects, the buffer has a pH below 7. In various aspects, the buffer comprises a weak acid, conjugate base, and a salt. In various aspects, the buffer is phosphate buffered saline (PBS). The concentration of the primary salt in the DF solution is in some aspects based on the concentration of the oligonucleotide compound present in the starting solution. For instance, the molar ratio of the oligonucleotide compound of the starting solution to the primary salt of the DF solution is about 1:2 to about 1:100 or about 1:2 to about 1:90 or about 1:2 to about 1:80. In various aspects, the molar ratio of the oligonucleotide compound of the starting solution to the primary salt of the DF solution is about 1:3 to about 1:80 or about 1:3 to about 1:75 or about 1:3 to about 1:70. Optionally, the molar ratio of the oligonucleotide compound of the starting solution to the primary salt of the DF solution is about 1:5 to about 1:65. For example, the molar ratio is about 1:5 to about 1:60, about 1:5 to about 1:55, about 1:5 to about 1:50, about 1:5 to about 1:45, about 1:5 to about 1:40, about 1:5 to about 1:35, about 1:5 to about 1:30, about 1:5 to about 1:25, about 1:5 to about 1:20, about 1:5 to about 1:15, about 1:5 to about 1:10, about 1:6 to about 1:65, about 1:7 to about 1:65, about 1:8 to about 1:65, about 1:9 to about 1:65, about 1:10 to about 1:65, about 1:15 to about 1:65, about 1:20 to about 1:65, about 1:25 to about 1:65, about 1:30 to about 1:65, about 1:35 to about 1:65, about 1:40 to about 1:65, about 1:45 to about 1:65, about 1:50 to about 1:65, about 1:55 to about 1:65, or about 1:60 to about 1:65. In various instances, the concentration of the primary salt is greater than 50% of the total salt concentration of the DF solution, optionally, greater than 55% or greater than 60% or greater than 65% or greater than 70% or greater than 75% or greater than 80% of the total salt concentration. In various instances, the concentration of the primary salt is greater than 80% of the total salt concentration of the DF solution, optionally, greater than 85% or greater than 90% of the total salt concentration.

The primary salt may be any salt. In various aspects, the primary salt comprises a monovalent cation. Optionally, the monovalent cation is sodium. In various aspects, the primary salt is sodium chloride. In various instances, the primary salt comprises a divalent cation. Optionally, the divalent cation is magnesium or calcium. Optionally, the primary salt is magnesium chloride or calcium chloride.

The DF solution in exemplary instances comprises the primary salt in an amount that causes the hydrodynamic diameter of the oligonucleotide compound to increase by at least 1.5 times or at least 2 times or at least 3 times compared to the hydrodynamic diameter of the oligonucleotide compound in water. Optionally, the DF solution comprises the primary salt in an amount that causes the hydrodynamic diameter of the oligonucleotide compound to increase 2-times, compared to the hydrodynamic diameter of the oligonucleotide compound in water. In various instances, the DF solution comprises the primary salt in an amount that causes the hydrodynamic diameter of the oligonucleotide compound to be about 5 nm to about 6 nm. In various aspects, the DF solution comprises the primary salt in an amount that causes the melting temperature of the oligonucleotide compound to increase by at least 1.5 times of at least 2 times compared to the melting temperature of the oligonucleotide compound in water. For instance, the melting temperature increases by about 20 degrees or about 30 degrees.

Also provided herein are methods of concentrating by ultrafiltration a first solution comprising a low concentration of an oligonucleotide compound to obtain a second solution comprising a high concentration of the oligonucleotide compound, wherein the oligonucleotide compound concentration of the second solution is greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL. In exemplary aspects, the oligonucleotide compound concentration of the first solution is less than about 140 mg/mL, less than about 130 mg/mL, less than about 120 mg/mL, less than about 110 mg/mL, less than about 100 mg/mL, less than about 90 mg/mL, less than about 80 mg/mL, less than about 70 mg/mL, less than about 60 mg/mL, or less than about 50 mg/mL. In various aspects, the second solution is a retentate obtained upon ultrafiltration. In exemplary instances, the retentate is obtained within 12 hours of ultrafiltration. In exemplary aspects, the duration of the ultrafiltration is less than 12 hours, less than 10 hours, less than 8 hours, less than 6 hours, less than 4 hours, less than 2 hours, or less than 1 hour. In various aspects, the retentate is achieved after 1 or 2 hours of ultrafiltration and the concentration of the oligonucleotide compound is greater than 150 mg/mL, optionally, greater than 175 mg/mL, greater than 200 mg/mL, or greater than 225 mg/mL. In exemplary instances, the oligonucleotide compound is double stranded. In exemplary instances, the total salt concentration of the first solution is about 25 mM to about 800 mM. Optionally, the total salt concentration of the first solution is about 25 mM to about 500 mM or about 25 mM to about 250 mM. In various aspects, the first solution comprises only one salt. Optionally, the one salt is an inorganic salt, such as, any of the inorganic salts described herein. In various aspects, the first solution does not comprise any acetate. In exemplary instances, the method of concentrating is carried out after diafiltration with a DF solution. In various instances, the diafiltration is carried out with a DF solution comprising one or more salts and the total salt concentration of the DF solution is about 25 mM to about 800 mM. Optionally, the total salt concentration of the DF solution is about 25 mM to about 500 mM or about 25 mM to about 250 mM. In exemplary aspects, the method of concentrating is carried out before diafiltration with a DF solution. In various instances, the oligonucleotide compound is a double-stranded oligonucleotide compound, optionally, an siRNA.

### Additional Steps

The methods disclosed herein, in various aspects, comprise additional steps. For example, in some aspects, the methods comprise one or more upstream steps or downstream steps involved in preparing a drug substance and/or a drug product. Optionally, the downstream steps are any one of those downstream processing steps described herein or known in the art. In exemplary embodiments, the method comprises steps for producing the oligonucleotide compound, including for example, conventional nucleic acid solid phase synthesis. The oligonucleotide compound can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA). The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides. The various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing oligonucleotides are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

In exemplary embodiments, the methods disclosed herein comprise steps for isolating and/or purifying the oligonucleotide compound. “Purify” or “purification” refers to a process that reduces the amounts of substances that are different than the target molecule (e.g. oligonucleotide compound) and are desirably excluded from the final liquid composition or preparation. Purification may refer to removal of one or more impurities. The term “impurity” refers to a substance having a different structure than the target molecule and the term can include a single undesired substance or a combination of several undesired substances. Impurities can include materials or reagents used in the methods to produce the oligonucleotide compounds as well as fragments or other undesirable derivatives or forms of the oligonucleotide compounds. In certain embodiments, the impurities comprise one or more oligonucleotides having a shorter length than the target oligonucleotide compound. In these and other embodiments, the impurities comprise one or more failure sequences. Failure sequences can be generated during the synthesis of the target oligonucleotide and arise from the failure of coupling reactions during the stepwise addition of a nucleotide monomer to the oligonucleotide chain. The product of an oligonucleotide synthetic reaction is often a heterogeneous mixture of oligonucleotides of varying lengths comprising the target oligonucleotide and various failure sequences having lengths shorter than the target oligonucleotide (i.e., truncated versions of the target oligonucleotide). In some embodiments, the impurities comprise one or more process-related impurities. Depending on the synthetic method to produce the oligonucleotide compound, such process-related impurities can include, but are not limited to, nucleotide monomers, protecting groups, salts, enzymes, and endotoxins. In exemplary aspects, the method comprises one or more chromatography steps for separating molecular species of a mixture comprising the oligonucleotide compound. In exemplary instances, the chromatography is analytical chromatography. In other exemplary instances, the chromatography is preparative chromatography. In exemplary aspects, each molecular species of the mixture is separated by way of the time at which it elutes from the matrix. In various instances, each molecular species of the mixture elutes at a time distinct from the time at which a different molecular species elutes. In various aspects, the molecular species are separated by reversed phase-high performance liquid chromatography (RP-HPLC). Reversed phased chromatography, e.g., RP-HPLC, is described in great detail in the prior art. See, for instance, Reversed Phase Chromatography: Principles and Methods, ed. AA, Amersham Biosciences, Buckinghamshire, England (1999).

The methods of the disclosure, in various aspects, comprise one or more steps for preparing the high concentration liquid composition obtained by the methods of the present disclosure into a drug product. In various aspects, the method comprises one or more formulation steps. Formulation is the process that transitions a drug substance into a formulated drug product. In various aspects, the method comprises one or more formulation steps to bring the liquid composition obtained by the presently disclosed method from an environment, solvent or other physical state into a form suitable for clinical administration.

In various aspects, the method of preparing a high concentration, liquid composition comprising greater than 150 mg/mL oligonucleotide compound further comprises an additional diafiltration or buffer exchange step, wherein the oligonucleotide compound is exchanged into a new solution, such as a formulation buffer. In exemplary instances, the method comprises (i) exchanging by diafiltration an oligonucleotide compound in a starting solution into a diafiltration (DF) solution to obtain an intermediate solution, wherein the concentration of the oligonucleotide compound in the starting solution is 140 mg/ml or less and the DF solution comprises one or more salts and the total salt concentration of the DF solution is about 25 mM to about 800 mM, optionally, less than 500 mM, (ii) concentrating by ultrafiltration the oligonucleotide compound in the intermediate solution to obtain a high concentration, liquid composition, wherein the concentration of the oligonucleotide compound in the high concentration, liquid composition is about 150 mg/mL or greater, and (iii) exchanging by diafiltration the oligonucleotide compound in the high concentration, liquid composition into a second DF solution. In various aspects, the second DF solution is a formulation buffer and the method achieves a high concentration, liquid formulation comprising greater than 150 mg/mL oligonucleotide compound. In various aspects, the method further comprises storing the high concentration, liquid formulation comprising greater than 150 mg/mL oligonucleotide compound at a temperature below 0 degrees C. to obtain a frozen preparation.

In various aspects, the method comprises carrying out one or more washes to free any oligonucleotide compound from the membrane. In various aspects, the method comprises removing the high concentration liquid composition from a collection vessel, e.g., a retentate vessel, and then carrying out one or more washes to free any oligonucleotide compound from the membrane. In various instances, the oligonucleotide compound obtained from the one or more washes is combined with the high concentration liquid composition obtained from the collection vessel, e.g., retentate vessel. In various aspects, the oligonucleotide compound obtained from the one or more washes combined with the high concentration liquid composition obtained from the collection vessel, e.g., retentate vessel, represents a greater than 70% recovery, e.g., greater than 70% of the oligonucleotide compound of the starting solution. In various instances, the oligonucleotide compound obtained from the one or more washes combined with the high concentration liquid composition obtained from the collection vessel, e.g., retentate vessel, comprises a concentration of about 150 mg/ml or greater.

In exemplary aspects, the additional steps do not involve lyophilization or freeze-drying of the oligonucleotide compound. Advantageously, the methods of the present invention provide a high concentration, liquid composition comprising at least 150 mg/mL oligonucleotide compound such that lyophilization or freeze-drying of the oligonucleotide compound is not needed and is avoided.

In various aspects, the oligonucleotide compound is a double-stranded (ds) oligonucleotide compound comprising an antisense strand and sense strand. In various aspects, the method of the present disclosure comprises annealing the sense strand and antisense strand to obtain the ds oligonucleotide compound and then preparing a high concentration, liquid composition comprising the ds oligonucleotide compound as described herein by (i) exchanging by diafiltration the ds oligonucleotide compound in a starting solution into a diafiltration (DF) solution to obtain an intermediate solution, wherein the concentration of the oligonucleotide compound in the starting solution is 140 mg/mL or less and the DF solution comprises one or more salts and the total salt concentration of the DF solution is about 25 mM to about 800 mM, optionally, less than 500 mM, and (ii) concentrating by ultrafiltration the ds oligonucleotide compound in the intermediate solution to obtain a high concentration, liquid composition. In various aspects, the starting solution is the solution obtained from annealing. In various aspects, the ds oligonucleotide compound is present in the starting solution at a concentration of about 50 mg/mL.

### Liquid Compositions and Frozen Preparations

The present disclosure also provides a liquid composition prepared by the method of the present disclosure. In various aspects, the liquid composition is equivalent to the high concentration liquid composition, e.g., the final retentate. In various instances, the liquid composition prepared by the method of the present disclosure comprises greater than or about 150 mg/ml of the oligonucleotide compound. Optionally, the concentration of the oligonucleotide compound in the liquid composition is at least 155 mg/mL, at least 160 mg/mL, at least 165 mg/mL, at least 170 mg/mL, at least 175 mg/mL, at least 180 mg/mL, at least 185 mg/mL, at least 190 mg/mL, at least 195 mg/mL, or at least 200 mg/mL. In various instances, the oligonucleotide compound is present in the liquid composition at a concentration of at least 210 mg/mL, at least 220 mg/mL, at least 230 mg/mL, of higher. The present disclosure also provides a frozen preparation made by storing the presently disclosed liquid composition at a temperature below 0 degrees C. The frozen preparation in exemplary aspects is not a lyophilized or freeze dried preparation.

Advantageously, the liquid composition prepared by the method is storage-stable and/or the liquid composition is stable against one or more freeze-thaw cycles. In various aspects, after storage at a temperature less than or equal to 0 degrees C. the liquid composition comprises at least 95% of the oligonucleotide compound as determined by HPLC. In various aspects, after storage at a temperature less than or equal to 0 degrees C. for at least about 1 week, at least about 2 weeks or at least about 4 weeks, the liquid composition comprises at least 95% of the oligonucleotide compound as determined by HPLC. In exemplary instances, after storage at a temperature less than or equal to 0 degrees C. for at least about 1 week or about 2 weeks or about 4 weeks followed by a thaw to 2-8 degrees C., the liquid composition comprises at least 95% of the oligonucleotide compound as determined by HPLC. In exemplary instances, after one or more freeze-thaw cycles, the liquid composition comprises at least 95% of the oligonucleotide compound as determined by HPLC.

In various aspects, the oligonucleotide compound of the liquid composition has not been subjected to lyophilization or freeze-drying. In aspects of the presently disclosed liquid compositions, the oligonucleotide compound has not been lyophilized or freeze-dried. The liquid composition is not a reconstituted lyophilized oligonucleotide compound drug substance or drug product. Optionally, throughout the method of manufacturing, the oligonucleotide compound is always in solution or in a frozen solution.

### Manufacturing Methods

The present disclosure further provides methods of manufacturing a solution medicament comprising an oligonucleotide compound or solution API (a solution of an oligonucleotide API). In exemplary aspects, the presently disclosed methods of manufacturing do not include any lyophilization. In exemplary instances of the presently disclosed methods of manufacturing, the API is not subjected to lyophilization. In exemplary embodiments, the method comprises carrying out the method of the present disclosure to obtain a high concentration, liquid composition comprising greater than about 150 mg/ml of the oligonucleotide compound, formulating the high concentration, liquid composition with a pharmaceutically-acceptable excipient, and filling the formulated high concentration, liquid composition into a container. The pharmaceutically-acceptable excipient may be any of those described in The Handbook of Pharmaceutical Excipients, Third Edition, A. H. Kibbe (Pharmaceutical Press, London, UK, 2000), or Remington's Pharmaceutical Sciences, Sixteenth Edition, E. W. Martin (Mack Publishing Co., Easton, Pa., 1980), each of which is incorporated by reference in its entirety.

In exemplary embodiments, the method comprises (a) preparing an oligonucleotide compound in a first solution comprising one more salts, wherein the concentration of the oligonucleotide compound in the solution is 140 mg/ml or less and the total salt concentration of the solution is about 25 mM to about 800 mM, and (b) concentrating by ultrafiltration the first solution to obtain a high concentration, liquid composition comprising the oligonucleotide compound at a concentration of about 150 mg/ml or greater. In various aspects, the first solution is prepared by diafiltration with a DF solution. In various instances, the diafiltration achieves an exchange of a starting solution comprising the oligonucleotide compound at about 140 mg/mL or less into a DF solution comprising one or more salts and having a total salt concentration of 25 mM to about 800 mM. Accordingly, in exemplary embodiments, the method of preparing the high concentration liquid composition comprises: (a) exchanging by diafiltration an oligonucleotide compound in a starting solution into a DF solution to obtain an intermediate solution, wherein the concentration of the oligonucleotide compound in the starting solution is 140 mg/mL or less and the DF solution comprises one or more salts, and (b) concentrating by ultrafiltration the oligonucleotide compound in the intermediate solution to obtain a high concentration, liquid composition, wherein the concentration of the oligonucleotide compound in the high concentration, liquid composition is about 150 mg/mL or greater. In exemplary instances, the total salt concentration of the DF solution is about 25 mM to about 800 mM. Optionally, the total salt concentration of the DF solution is about 25 mM to about 500 mM. In exemplary instances, the method comprises continuous diafiltration and/or ultrafiltration by tangential flow filtration. In various aspects, the method employs a polyethersulfone (PES) membrane or a stabilized cellulose membrane for the diafiltration and/or ultrafiltration. Optionally, the membrane has a molecular weight cut-off (MWCO) of less than 10 kDa, and in exemplary instances, the MWCO is about 5 kDa or about 3 kDa.

In exemplary embodiments, the method comprises concentrating by ultrafiltration a first solution comprising a low concentration of an oligonucleotide compound to obtain a second solution comprising a high concentration of the oligonucleotide compound, wherein the oligonucleotide compound concentration of the second solution is greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL. In exemplary aspects, the oligonucleotide compound concentration of the first solution is less than about 140 mg/mL, less than about 130 mg/mL, less than about 120 mg/mL, less than about 110 mg/mL, less than about 100 mg/mL, less than about 90 mg/mL, less than about 80 mg/mL, less than about 70 mg/mL, less than about 60 mg/mL, or less than about 50 mg/mL. In various aspects, the second solution is a retentate obtained upon ultrafiltration. In various aspects, the retentate is achieved after 1 or 2 hours of ultrafiltration. In exemplary instances, the oligonucleotide compound is double stranded. In exemplary instances, the total salt concentration of the first solution is about 25 mM to about 800 mM. Optionally, the total salt concentration of the first solution is about 25 mM to about 500 mM or about 25 mM to about 250 mM. In various aspects, the first solution comprises only one salt. Optionally, the one salt is an inorganic salt, such as, any of the inorganic salts described herein. In various aspects, the first solution does not comprise any acetate. In exemplary instances, the method of concentrating is carried out after diafiltration with a DF solution. In various instances, the diafiltration is carried out with a DF solution comprising one or more salts and the total salt concentration of the DF solution is about 25 mM to about 800 mM. Optionally, the total salt concentration of the DF solution is about 25 mM to about 500 mM or about 25 mM to about 250 mM. In exemplary aspects, the method of concentrating is carried out before diafiltration with a DF solution. In various instances, the oligonucleotide compound is a double-stranded oligonucleotide compound, optionally, an siRNA.

Further provided herein is a method of preparing a solution API comprising an oligonucleotide compound API, wherein the oligonucleotide compound API is present in the solution at a concentration greater than 150 mg/mL. In various aspects, the oligonucleotide compound API is double-stranded, optionally, an siRNA. In exemplary embodiments, the method of preparing a solution API comprises a presently disclosed method of concentrating by ultrafiltration a first solution comprising a low concentration of an oligonucleotide compound to obtain a second solution comprising a high concentration of the oligonucleotide compound. In various instances, the method of preparing a solution API comprises (i) synthesizing the oligonucleotide compound, or a strand thereof, by, e.g., solid phase synthesis, (ii) carrying out one or more of a chromatography, a diafiltration, and an annealing, and (iii) concentrating by ultrafiltration in accordance with a presently disclosed method of concentrating by ultrafiltration a first solution comprising a low concentration of an oligonucleotide compound to obtain a second solution comprising a high concentration of the oligonucleotide compound. In various aspects, the oligonucleotide compound concentration of the second solution is greater than about 150 mg/mL, greater than about 160 mg/mL, greater than about 170 mg/mL, greater than about 180 mg/mL, greater than about 190 mg/mL, greater than about 200 mg/mL, greater than about 210 mg/mL, greater than about 220 mg/mL, and the oligonucleotide compound concentration of the first solution is less than about 140 mg/mL, less than about 130 mg/mL, less than about 120 mg/mL, less than about 110 mg/mL, less than about 100 mg/mL, less than about 90 mg/mL, less than about 80 mg/mL, less than about 70 mg/mL, less than about 60 mg/mL, or less than about 50 mg/mL. In various aspects, the second solution is a retentate obtained upon ultrafiltration. In various aspects, the retentate is achieved after 1 or 2 hours of ultrafiltration. In exemplary aspects of the method of preparing a solution API comprising an oligonucleotide compound API, the oligonucleotide API is not lyophilized at any point during the method. Advantageously, the method of preparing the solution API is a method which lacks any lyophilization. Accordingly, the method of preparing the solution API of the present disclosure is free of a lyophilization and provides a more time- and cost-efficient way of preparing an oligonucleotide compound API. In various instances, the solution API prepared by the presently disclosed method is directly used for preparing a solution DP comprising the oligonucleotide compound API. Optionally, the solution API is sterile filtered and filled into vials or pre-filled syringes or autoinjectors. Accordingly, a method of producing a DP, e.g., a solution DP, comprising a solution API comprising an oligonucleotide compound API is provided. In various aspects, the method of producing the DP lacks any lyophilization. In various aspects of the method of producing the DP, lyophilization of the oligonucleotide compound API is not carried out at any time during the method.

In various instances, the method of manufacturing conforms to the current good manufacturing practices (cGMP) recommended by the U.S. Food and Drug Administration. Accordingly, the presently disclosed method of manufacturing leads to a cGMP grade medicament comprising a high concentration (greater than about 150 mg/ml) of the oligonucleotide compound and the medicament is a liquid composition. In various aspects, the method of manufacturing comprises carrying out the method of the present disclosure to obtain a cGMP grade, high concentration, liquid composition comprising greater than about 150 mg/mL of the oligonucleotide compound. In exemplary aspects, the medicament is a clinical grade, high concentration liquid composition comprising greater than about 150 mg/ml of the oligonucleotide compound. In aspects of the presently disclosed methods of manufacture, the oligonucleotide compound is not subjected to any lyophilization or freeze-drying. In aspects of the presently disclosed methods of manufacture, the oligonucleotide compound is not lyophilized or freeze-dried. Optionally, throughout the method of manufacturing, the oligonucleotide compound is always in solution or in a frozen solution.

In various aspects, the oligonucleotide compound is a double-stranded (ds) oligonucleotide compound comprising an antisense strand and sense strand. In various aspects, the method of manufacturing comprises annealing the sense strand and antisense strand to obtain the ds oligonucleotide compound and then preparing a high concentration, liquid composition as described herein by (i) exchanging by diafiltration the ds oligonucleotide compound in a starting solution into a diafiltration (DF) solution to obtain an intermediate solution, wherein the concentration of the oligonucleotide compound in the starting solution is 140 mg/mL or less and the DF solution comprises one or more salts and the total salt concentration of the DF solution is about 25 mM to about 800 mM, optionally, less than 500 mM, and (ii) concentrating by ultrafiltration the ds oligonucleotide compound in the intermediate solution to obtain a high concentration, liquid composition.

In various aspects, the method further comprises finishing the final retentate, optionally, further comprising filling the finished final retentate into a container. In various aspects, the method further comprises aseptic filtration of the drug substance prior to storage. In various instances, the method is completely devoid of a lyophilization of the oligonucleotide compound.

### Oligonucleotides and Oligonucleotide Compounds

As used herein, oligonucleotide compounds represent a class of biochemical entities comprising one or more oligonucleotides, wherein an oligonucleotide is an oligomer or polymer of nucleotides or modified nucleotides. For instance, an oligonucleotide may comprise ribonucleotides, deoxyribonucleotides, modified nucleotides, or combinations thereof. Optionally, the oligonucleotide comprises nucleotides or modified nucleotides linked to one another by phosphodiester linkages and/or modified internucleotide linkages. The oligonucleotide compound, in various instances, comprises only one oligonucleotide, while in other instances, the oligonucleotide compound comprises two, three, four, or more oligonucleotides. The oligonucleotide may be, for instance, a duplex, triplex, or a quadruplex comprising two, three, and four oligonucleotides, respectively. In various aspects, the oligonucleotide compound comprises two or more oligonucleotides and each is linked to another by non-covalent bonds. In various aspects, the oligonucleotide compound is a duplex comprising an oligonucleotide its complement strand and each oligonucleotide is bound to the other via hydrogen bonds. The oligonucleotide compound, in various instances, is single-stranded or double-stranded.

Oligonucleotide components of oligonucleotide compounds can be a few nucleotides in length up to several hundred nucleotides in length, for example, from about 10 nucleotides in length to about 300 nucleotides in length, from about 12 nucleotides in length to about 100 nucleotides in length, from about 15 nucleotides in length to about 250 nucleotides in length, from about 20 nucleotides in length to about 80 nucleotides in length, from about 15 nucleotides in length to about 30 nucleotides in length, from about 18 nucleotides in length to about 26 nucleotides in length, or from about 19 nucleotides in length to about 23 nucleotides in length. In some embodiments, the oligonucleotide component(s) of an oligonucleotide compound used in the methods of the invention is about 18, 19, 20, 21, 22, 23, 24, 25, or 26 nucleotides in length. In one embodiment, the oligonucleotide is about 19 nucleotides in length. In another embodiment, the oligonucleotide is about 20 nucleotides in length. In yet another embodiment, the oligonucleotide is about 21 nucleotides in length. In still another embodiment, the oligonucleotide is about 23 nucleotides in length.

The oligonucleotide compound may be a naturally occurring oligonucleotide isolated from a cell or organism. For instance, the oligonucleotide compound may be derived from or a fragment of genomic DNA, particularly the telomere or promoter regions, or may be derived from or a fragment of messenger RNA (mRNA), particularly the 5′ or 3′ untranslated regions. In some embodiments, the oligonucleotide compound is a synthetic oligonucleotide compound produced by chemical synthetic methods or in vitro enzymatic methods. In some embodiments oligonucleotides are single stranded RNA or DNA. In some embodiments, the oligonucleotide compound is a short hairpin RNA (shRNA), a precursor miRNA (pre-miRNA), an anti-miRNA oligonucleotide (e.g. antagomir and antimiR), or an antisense oligonucleotide. In other embodiments, the oligonucleotide compound is a double-stranded RNA molecule or RNA interference agent, such as a small interfering RNA (siRNA), a microRNA (miRNA), or a miRNA mimetic.

In certain embodiments, the oligonucleotide compound is a therapeutic oligonucleotide designed to target a gene or RNA molecule associated with a disease or disorder. For instance, in one embodiment, the oligonucleotide compound is an antisense oligonucleotide (ASO) that comprises a single strand sequence complementary to a region of a target gene or mRNA sequence. A first sequence is “complementary” to a second sequence if an oligonucleotide comprising the first sequence can hybridize to an oligonucleotide comprising the second sequence to form a duplex region under certain conditions. “Hybridize” or “hybridization” refers to the pairing of complementary oligonucleotides, typically via hydrogen bonding (e.g. Watson-Crick, Hoogsteen or reverse Hoogsteen hydrogen bonding) between complementary bases in the two oligonucleotides. A first sequence is considered to be fully complementary (100% complementary) to a second sequence if an oligonucleotide comprising the first sequence base pairs with an oligonucleotide comprising the second sequence over the entire length of one or both nucleotide sequences without any mismatches.

In another embodiment, the oligonucleotide compound is an siRNA or other type of double-stranded RNA interference agent comprising an antisense strand and a sense strand, wherein the antisense strand comprises a sequence that is complementary to a region of a target gene or mRNA sequence. The strand of an siRNA or other type of double-stranded RNA interference agent comprising a region having a sequence that is complementary to a target sequence (e.g. target mRNA) is referred to as the “antisense strand.” The “sense strand” refers to the strand that includes a region that is complementary to a region of the antisense strand. In some embodiments, the sense strand may comprise a sequence that is identical to a region of a target gene or mRNA sequence.

The oligonucleotide compound may comprise one or more modified nucleotides. A “modified nucleotide” refers to a nucleotide that has one or more chemical modifications to the nucleoside, nucleobase, pentose ring, or phosphate group. Such modified nucleotides can include, but are not limited to, nucleotides with 2′ sugar modifications (2′-O-methyl, 2′-methoxyethyl, 2′-fluoro, deoxynucleotides, etc.), abasic nucleotides, inverted nucleotides (3′-3′ linked nucleotides), phosphorothioate linked nucleotides, nucleotides with bicyclic sugar modifications (e.g. LNA, ENA), and nucleotides comprising base analogs (e.g. universal bases, 5-methylcytosine, pseudouracil, etc.).

In certain embodiments, the modified nucleotides have a modification of the ribose sugar. These sugar modifications can include modifications at the 2′ and/or 5′ position of the pentose ring as well as bicyclic sugar modifications. A 2′-modified nucleotide refers to a nucleotide having a pentose ring with a substituent at the 2′ position other than OH. Such 2′-modifications include, but are not limited to, 2′-H (e.g. deoxyribonucleotides), 2′-O-alkyl (e.g. O—C₁-C₁₀ or O—C₁-C₁₀ substituted alkyl), 2′-O-allyl (O—CH₂CH═CH₂), 2′-C-allyl, 2′-fluoro, 2′-O-methyl (OCH₃), 2′-O-methoxyethyl (O—(CH₂)₂OCH₃), 2′-OCF₃, 2′-O(CH₂)₂SCH₃, 2′-O-aminoalkyl, 2′-amino (e.g. NH₂), 2′-O-ethylamine, and 2′-azido. Modifications at the 5′ position of the pentose ring include, but are not limited to, 5′-methyl (R or S); 5′-vinyl, and 5′-methoxy. A “bicyclic sugar modification” refers to a modification of the pentose ring where a bridge connects two atoms of the ring to form a second ring resulting in a bicyclic sugar structure. In some embodiments the bicyclic sugar modification comprises a bridge between the 4′ and 2′ carbons of the pentose ring. Nucleotides comprising a sugar moiety with a bicyclic sugar modification are referred to herein as bicyclic nucleic acids or BNAs. Exemplary bicyclic sugar modifications include, but are not limited to, α-L-Methyleneoxy (4′-CH₂—O-2′) bicyclic nucleic acid (BNA); β-D-Methyleneoxy (4′-CH₂—O-2′) BNA (also referred to as a locked nucleic acid or LNA); Ethyleneoxy (4′-(CH₂)₂—O-2′) BNA; Aminooxy (4′-CH₂—O—N(R)-2′) BNA; Oxyamino (4′-CH₂—N(R)—O-2′) BNA; Methyl(methyleneoxy) (4′-CH(CH₃)—O-2′) BNA (also referred to as constrained ethyl or cEt); methylene-thio (4′-CH₂—S-2′) BNA; methylene-amino (4′-CH₂—N(R)-2′) BNA; methyl carbocyclic (4′-CH₂—CH(CH₃)-2′) BNA; propylene carbocyclic (4′-(CH₂)₃-2′) BNA; and Methoxy(ethyleneoxy) (4′-CH(CH₂OMe)-O-2′) BNA (also referred to as constrained MOE or cMOE). These and other sugar-modified nucleotides that can be incorporated into the oligonucleotide compounds are described in U.S. Pat. No. 9,181,551, U.S. Patent Publication No. 2016/0122761, and Deleavey and Damha, Chemistry and Biology, Vol. 19:937-954, 2012, all of which are hereby incorporated by reference in their entireties.

In some embodiments, the oligonucleotide compounds comprise one or more 2′-fluoro modified nucleotides, 2′-O-methyl modified nucleotides, 2′-O-methoxyethyl modified nucleotides, 2′-O-allyl modified nucleotides, bicyclic nucleic acids (BNAs), or combinations thereof. In certain embodiments, the oligonucleotide compounds comprise one or more 2′-fluoro modified nucleotides, 2′-O-methyl modified nucleotides, 2′-O-methoxyethyl modified nucleotides, or combinations thereof. In one particular embodiment, the oligonucleotide compounds comprise one or more 2′-fluoro modified nucleotides, 2′-O-methyl modified nucleotides, deoxynucleotides, or combinations thereof. In another particular embodiment, the oligonucleotide compounds comprise one or more 2′-fluoro modified nucleotides, 2′-O-methyl modified nucleotides, or combinations thereof.

The oligonucleotide compounds may also comprise one or more modified internucleotide linkages. As used herein, the term “modified internucleotide linkage” refers to an internucleotide linkage other than the natural 3′ to 5′ phosphodiester linkage. In some embodiments, the modified internucleotide linkage is a phosphorous-containing internucleotide linkage, such as a phosphotriester, aminoalkylphosphotriester, an alkylphosphonate (e.g. methylphosphonate, 3′-alkylene phosphonate), a phosphinate, a phosphoramidate (e.g. 3′-amino phosphoramidate and aminoalkylphosphoramidate), a phosphorothioate (P═S), a chiral phosphorothioate, a phosphorodithioate, a thionophosphoramidate, a thionoalkylphosphonate, a thionoalkylphosphotriester, and a boranophosphate. In one embodiment, a modified internucleotide linkage is a 2′ to 5′ phosphodiester linkage. In other embodiments, the modified internucleotide linkage is a non-phosphorous-containing internucleotide linkage and thus can be referred to as a modified internucleoside linkage. Such non-phosphorous-containing linkages include, but are not limited to, morpholino linkages (formed in part from the sugar portion of a nucleoside); siloxane linkages (—O—Si(H)₂—O—); sulfide, sulfoxide and sulfone linkages; formacetyl and thioformacetyl linkages; alkene containing backbones; sulfamate backbones; methylenemethylimino (—CH₂—N(CH₃)—O—CH₂—) and methylenehydrazino linkages; sulfonate and sulfonamide linkages; amide linkages; and others having mixed N, O, S and CH₂ component parts. In one embodiment, the modified internucleoside linkage is a peptide-based linkage (e.g. aminoethylglycine) to create a peptide nucleic acid or PNA, such as those described in U.S. Pat. Nos. 5,539,082; 5,714,331; and 5,719,262. Other suitable modified internucleotide and internucleoside linkages that may be incorporated into the oligonucleotide compounds are described in U.S. Pat. Nos. 6,693,187, 9,181,551, U.S. Patent Publication No. 2016/0122761, and Deleavey and Damha, Chemistry and Biology, Vol. 19:937-954, 2012, all of which are hereby incorporated by reference in their entireties.

In certain embodiments, the oligonucleotide compounds comprise one or more phosphorothioate internucleotide linkages. The oligonucleotide compounds may comprise 1, 2, 3, 4, 5, 6, 7, 8, or more phosphorothioate internucleotide linkages. In some embodiments, all of the internucleotide linkages in the oligonucleotide compounds are phosphorothioate internucleotide linkages. In other embodiments, the oligonucleotide compounds can comprise one or more phosphorothioate internucleotide linkages at the 3′-end, the 5′-end, or both the 3′- and 5′-ends. For instance, in certain embodiments, the oligonucleotide compounds comprise about 1 to about 6 or more (e.g., about 1, 2, 3, 4, 5, 6 or more) consecutive phosphorothioate internucleotide linkages at the 3′-end. In other embodiments, the oligonucleotide compounds comprise about 1 to about 6 or more (e.g., about 1, 2, 3, 4, 5, 6 or more) consecutive phosphorothioate internucleotide linkages at the 5′-end.

The oligonucleotide compounds to be used in the methods of the invention can readily be made using techniques known in the art, for example, using conventional nucleic acid solid phase synthesis. The oligonucleotides can be assembled on a suitable nucleic acid synthesizer utilizing standard nucleotide or nucleoside precursors (e.g. phosphoramidites). Automated nucleic acid synthesizers are sold commercially by several vendors, including DNA/RNA synthesizers from Applied Biosystems (Foster City, CA), MerMade synthesizers from BioAutomation (Irving, TX), and OligoPilot synthesizers from GE Healthcare Life Sciences (Pittsburgh, PA). The 2′ silyl protecting group can be used in conjunction with acid labile dimethoxytrityl (DMT) at the 5′ position of ribonucleosides to synthesize oligonucleotides via phosphoramidite chemistry. Final deprotection conditions are known not to significantly degrade RNA products. All syntheses can be conducted in any automated or manual synthesizer on large, medium, or small scale. The syntheses may also be carried out in multiple well plates, columns, or glass slides. The 2′-O-silyl group can be removed via exposure to fluoride ions, which can include any source of fluoride ion, e.g., those salts containing fluoride ion paired with inorganic counterions e.g., cesium fluoride and potassium fluoride or those salts containing fluoride ion paired with an organic counterion, e.g., a tetraalkylammonium fluoride. A crown ether catalyst can be utilized in combination with the inorganic fluoride in the deprotection reaction. Preferred fluoride ion sources are tetrabutylammonium fluoride or aminohydrofluorides (e.g., combining aqueous HF with triethylamine in a dipolar aprotic solvent, e.g., dimethylformamide). The various synthetic steps may be performed in an alternate sequence or order to give the desired compounds. Other synthetic chemistry transformations, protecting groups (e.g., for hydroxyl, amino, etc. present on the bases) and protecting group methodologies (protection and deprotection) useful in synthesizing oligonucleotides are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

As can be appreciated by the skilled artisan, further methods of synthesizing the oligonucleotide compounds will be evident to those of ordinary skill in the art. For instance, the oligonucleotides can be synthesized using enzymes in in vitro systems, such as in the methods described in Jensen and Davis, Biochemistry, Vol. 57:1821-1832, 2018. Naturally occurring oligonucleotides can be isolated from cells or organisms using conventional methods. Custom synthesis of oligonucleotides is also available from several commercial vendors, including Dharmacon, Inc. (Lafayette, CO), AxoLabs GmbH (Kulmbach, Germany), and Ambion, Inc. (Foster City, CA).

In various aspects, the oligonucleotide compound comprises or consists of the sequence of 5′-UCGUAUAACAAUAAGGGGCUG-3′ (SEQ ID NO: 2). In some such embodiments, the oligonucleotide compound comprises or consists of the sequence of modified nucleotides according to the sequence of 5′-usCfsgUfaUfaacaaUfaAfgGfgGfcsUfsg-3′ (SEQ ID NO: 4), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (“2′-fluoro”) adenosine, 2′-fluoro guanosine, 2′-fluoro cytidine, and 2′-fluoro uridine, respectively; and s is a phosphorothioate linkage. In various instances, a complementary oligonucleotide of the oligonucleotide compound comprises or consists of the sequence of 5′-CAGCCCCUUAUUGUUAUACGA-3′ (SEQ ID NO: 1). In related embodiments, the complementary oligonucleotide comprises or consists of the sequence of modified nucleotides according to the sequence of 5′-csagccccuUfAfUfuguuauacgs (invdA)-3′ (SEQ ID NO: 3), wherein a, g, c, and u are 2′-O-methyl adenosine, 2′-O-methyl guanosine, 2′-O-methyl cytidine, and 2′-O-methyl uridine, respectively; Af, Gf, Cf, and Uf are 2′-deoxy-2′-fluoro (“2′-fluoro”) adenosine, 2′-fluoro guanosine, 2′-fluoro cytidine, and 2′-fluoro uridine, respectively; invdA is an inverted deoxyadenosine (3′-3′ linked nucleotide), and s is a phosphorothioate linkage. In various instances, the oligonucleotide compound comprises a duplex comprising a sense strand and an antisense strand.

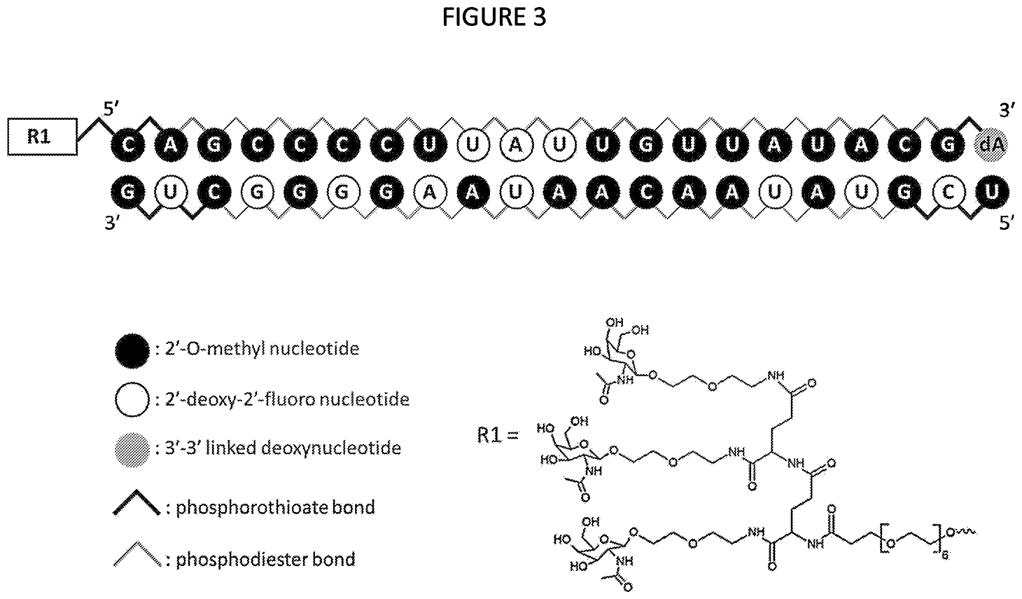

In some embodiments, the oligonucleotide compound may comprise additional components other than the oligonucleotide components. For instance, the oligonucleotide component of an oligonucleotide compound may be covalently linked to a sugar, a polymer, an amino acid, a fatty acid, a cholesterol moiety, a vitamin, a steroid, a folate moiety, peptide, polypeptide or protein. In certain embodiments, the oligonucleotide component of an oligonucleotide compound is covalently linked to a targeting moiety that delivers the oligonucleotide compound to a particular tissue or cell type. The targeting moiety may comprise an antibody or antigen-binding fragment thereof that specifically binds to a receptor expressed on the surface of a target cell type (e.g. hepatocytes). Alternatively, the targeting moiety may comprise a ligand for a receptor expressed on the surface of a target cell or tissue to which the oligonucleotide compound is to be delivered. In one such embodiment, the targeting moiety comprises a ligand for the asialoglycoprotein receptor (ASGPR), which is expressed on the surface of hepatocytes. Ligands of the ASGPR may comprise galactose, galactosamine, or N-acetyl-galactosamine (GalNAc). In certain embodiments, the ASGPR ligand comprises a multivalent galactose or multivalent GalNAc moiety, such as a trivalent or tetravalent galactose or GalNAc moiety. Such multivalent galactose and GalNAc moieties are known to those of skill in the art. An exemplary GalNAc moiety that can be covalently linked to an oligonucleotide compound used in the methods of the invention is shown in FIG. 3 as R1.

In certain embodiments, the oligonucleotide compound comprises a sense strand comprising the sequence of modified nucleotides according to SEQ ID NO: 3 and an antisense strand comprising the sequence of modified nucleotides according to SEQ ID NO: 4. The structure of an exemplary oligonucleotide compound is shown in FIG. 3 and is further described in the Examples. In exemplary instances, the oligonucleotide compound is double stranded and/or comprises a double helix structure. Optionally, the oligonucleotide compound is an siRNA. In various instances, the oligonucleotide is double stranded. In various aspects, the oligonucleotide compound comprises a duplex comprising an antisense oligonucleotide and a sense oligonucleotide. In exemplary aspects, each of the antisense and sense oligonucleotides independently comprises at least 11 nt, optionally, about 15 to about 30 nucleotides, about 18 to about 26 nucleotides, about 19 to about 23 nucleotides, or about 19 to about 21 nucleotides. Optionally, the oligonucleotide compound has a molecular weight greater than about 7,000 daltons or greater than about 10,000 daltons or greater than about 15,000 daltons. In certain embodiments, the oligonucleotide compound is a single-stranded antisense oligonucleotide. In such embodiments, the antisense oligonucleotide comprises about 15 to about 25 nucleotides, about 18 to about 22 nucleotides, or about 20 nucleotides.

In various aspects, the oligonucleotide compound modulates gene expression through a range of processes, including RNAi, target degradation by RNase H-mediated cleavage, splicing modulation, non-coding RNA inhibition, gene activation and programmed gene editing. Optionally, the oligonucleotide compound is any one of those described in Roberts et al., Nature Reviews Drug Discovery 19:673-694 (2020). In exemplary aspects, the oligonucleotide is an ASO. In various instances, the ASO is a single strand of about 18 to about 30 nucleotides. Optionally, the oligonucleotide compound is selected from the group consisting of: mipomersen, pegaptanib, defibrotide, patisiran, custirsen, fomivirsen, oblimersen, eteplirsen, nusinersen, pelacarsen, inotersen, givosiran, golodirsen, and viltolarsen. Optionally, the oligonucleotide compound is any of those described in Table 2 of Roberts et al., 2020, supra, such as, inclisiran, tivanisiran, miravirsen, GF012, TF-101, cobomarsen, remlarsen, SLN124, MTL-CEPBA, suvodirsen, fitusiran, lumasiran, vutrisiran, revusiran, casimersen, tofersen, and the like. In various aspects, the oligonucleotide is a double-stranded siRNA, such as, patisiran, givosiran, olpasiran.

### Methods of Treatment

Methods of treating a subject with a disease are furthermore provided. In exemplary embodiments, the method comprises administering to the subject a liquid composition of the present disclosure in an amount effective to treat the disease in the subject. Optionally, the medicament is administered by injection or infusion. As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating of the present disclosure can provide any amount or any level of treatment. Furthermore, the treatment provided by the method of the present disclosure can include treatment of one or more conditions or symptoms or signs of the disease being treated. Also, the treatment provided by the methods of the present disclosure can encompass slowing the progression of the disease. For example, the methods can treat the disease by virtue of reducing signs and symptoms of the disease, halt or slow the progression of the disease, reduce the frequency of the recurrence of the disease, delay the onset of the disease, and the like. In exemplary aspects, the methods treat by way of delaying the onset or recurrence of the disease by 1 day, 2 days, 4 days, 6 days, 8 days, 10 days, 15 days, 30 days, two months, 4 months, 6 months, 1 year, 2 years, 4 years, or more.

In various aspects, the disease, condition, or disorder to be treated or ameliorated according to the methods of the invention is a disease, condition, or disorder associated with aberrant target gene expression or activity, for example, where overexpression of a gene product causes a pathological phenotype. Exemplary target genes which the oligonucleotide compounds comprised in the liquid compositions target include, but are not limited to, LPA, PNPLA3, ASGR1, F7, F12, FXI, APOCIII, APOB, APOL1, TTR, PCSK9, SCAP, MARC1, KRAS, CD274, PDCD1, C5, ALAS1, HAO1, LDHA, ANGPTL3, SERPINA1, AGT, HAMP, LECT2, EGFR, VEGF, KIF11, AT3, CTNNB1, HMGB1, HIF1A, and STAT3. Target genes may also include viral genes, such as hepatitis B and hepatitis C viral genes, human immunodeficiency viral genes, herpes viral genes, etc. In some embodiments, the target gene is a gene that encodes a human micro RNA (miRNA). In some embodiments, the disease is a cardiovascular disease, e.g., atherosclerotic cardiovascular disease. In other embodiments, the disease is cancer. In certain other embodiments, the disease is a liver disease, such as nonalcoholic fatty liver disease, nonalcoholic steatohepatitis, cirrhosis. In various instances, the subject is an adult human.

The following examples are given merely to illustrate the present invention and not in any way to limit its scope.

### EXAMPLES

### Example 1

This example describes the evaluation of different membranes for use in concentrating a solution comprising a drug substance via Ultrafiltration/Diafiltration (UF/DF).

The purpose of this study was to identify a membrane with a molecular weight cut-off (MWCO) suitable for concentrating a duplex siRNA in the retentate. In this study, a double-stranded siRNA molecule was used as a model oligonucleotide compound. Each strand of the siRNA is 21 nucleotides long and contains nucleotides with chemical modifications. A targeting ligand comprising N-acetylgalactosamine is linked to the 5′ end of the sense strand for selective liver targeting. The structure of the siRNA molecule is provided in FIG. 3.

A lab-scale UF/DF system was used for buffer exchange (via diafiltration) and concentration (via ultrafiltration) of an siRNA sample. The system was fitted with one of several different membrane types as described in Table 1. A starting solution comprising the siRNA (30 mg/mL) and water was exchanged into a diafiltration (DF) solution comprising 12 mM potassium phosphate, dibasic (K₂HPO₄), 8 mM potassium phosphate, monobasic (KH₂PO₄), 40 mM sodium chloride (NaCl), and water for injection. UV analysis (at 260 nm) was carried out to measure the siRNA duplex concentration in the final retentate. A summary of the MWCO membrane, starting solution, DF solution, and siRNA duplex concentration of the retentate is provided in Table 1. Observational notes from each run are also provided in Table 1.

TABLE 1

| | | | | Final Retentate | |
|---|---|---|---|---|---|
| | | | | siRNA | |
| Membrane | MWCO of | Starting | Diafiltration | Concentration | Observational |
| Type | Membrane | Solution | Solution | (mg/mL) | Notes |
| Millipore | 10 kDa | 30 mg/mL | 12 mM K₂HPO₄, | 0 | Large flow-through |
| Pellicon ®, | | siRNA in | 8 mM KH₂PO₄, | | of siRNA duplex in |
| 100 cm² | | Milli-Q | 40 mM NaCl, | | permeate, resulting |
| | | Water | and water for | | in poor recovery. |
| | | | injection | | |
| Sartocon | 2 kDa | 30 mg/mL | 12 mM K₂HPO₄, | 88 | Flux drop to zero |
| Slice 200 | | siRNA in | 8 mM KH₂PO₄, | | prevented further |
| Hydrosart ®, | | Milli-Q | 40 mM NaCl, | | concentration. Flow |
| 20 cm² | | Water | and water for | | rate Too slow |
| | | | injection | | |
| Sartocon | 5 kDa | 30 mg/mL | 12 mM K₂HPO₄, | 136 | Flux drop to zero |
| Slice 200 | | siRNA in | 8 mM KH₂PO₄, | | prevented further |
| Hydrosart ®, | | Milli-Q | 40 mM NaCl, | | concentration. |
| 20 cm² | | Water | and water for | | |
| | | | injection | | |
| Sartocon | 5 kDa | 30 mg/mL | 12 mM K₂HPO₄, | 109 | Insufficient volume |
| Slice 200 | | siRNA in | 8 mM KH₂PO₄, | | of starting solution, |
| Hydrosart | | Milli-Q | 40 mM NaCl, | | and flux drop prevented |
| membrane, | | Water | and water for | | further concentration. |
| 100 cm² | | | injection | | |

The siRNA molecule used in the experiments has a molecular weight of 16.3 kDa, and therefore a 10-kDa filter membrane was selected. However, after UF/DF, most of the siRNA was detected in the permeate, not the retentate. Therefore, using a 10 kDa MWCO membrane was not considered as a good option. Given the above results, membranes having a smaller MWCO (e.g., <10 kDa MWCO) were evaluated. Hydrosart membranes having a MWCO of 2 kDa or 5 kDa (each sized 20 cm²) were analyzed. As shown in Table 1, the membrane with the 5-kDa MWCO led to a higher retentate siRNA concentration, compared to that achieved with the membrane with the 2-kDa MWCO. The 2-kDa MWCO membrane also demonstrated a very slow flowrate (<4 mL/min). In both instances, the flux dropped to zero which prevented further concentration of the siRNA in the retentate. Another 5-kDa MWCO membrane (Sartocon Slice 200 Hydrosart membrane, size 100 cm²) was evaluated. The retentate siRNA concentration achieved with this membrane was 109 mg/mL. The lower concentration was likely due to a smaller volume of the starting solution used in this experiment, compared to the volume of the starting solution used with the other 5-kDa MWCO membrane of Table 1. Taken together, the results of this study suggested that a 5-kDa MWCO membrane demonstrated highest suitability for UF/DF of the siRNA molecule.

In all cases, regardless of the membrane used, a flux decline was observed which limited the siRNA concentration in the retentate. Without being bound to any particular theory, the decline in flux was a consequence of membrane fouling. Additional studies were carried out to address this issue.

### Example 2

This example describes the effect of the diafiltration (DF) solution salt concentration on the siRNA concentration in the retentate post-UF/DF.

To examine the influence of the DF solution salt concentration on the retentate siRNA concentration, a series of UF/DF runs were carried out to concentrate an siRNA drug substance solution. Briefly, an Ambr Crossflow, high-throughput, small scale UF/DF system fitted with a 5 kDa MWCO Sartorius Hydrosart membrane was used. The starting solution (˜25 mL) comprising 30 mg/ml of the same siRNA molecule used in Example 1 in water was fed into the UF/DF system. This starting solution was exchanged into a DF solution in 10 diavolumes. The DF solution was 1×PBS, 0.5×PBS, or water. 1×PBS comprised the following components: 2.67 mM potassium chloride (KCl), 1.47 mM potassium phosphate monobasic (KH₂PO₄), 137.93 mM sodium chloride (NaCl), and 8.06 mM sodium phosphate dibasic (Na₂HPO₄·7H₂O). 0.5× PBS was made by diluting 1×PBS with an equal volume of water. After diafiltration, the feeding of the diafiltration solution was ceased to initiate the ultrafiltration process which concentrates the siRNA in the retentate. The permeate solution was collected in a container separate from the retentate. When the retentate volume was decreased to approximately 5 mL or when the permeate flow stopped, the ultrafiltration process was complete. The retentate was then harvested and subjected to UV analysis (at 260 nm) to determine the siRNA duplex concentration. Table 2 summarizes the experimental conditions and results.

TABLE 2

| | | NaCl | | |
|---|---|---|---|---|
| | | Concentration | Total salt | Retentate |
| | | of DF | concentration | siRNA |
| Starting | DF | solution | of DF | Concentration |
| solution | solution | (mM) | solution | (mg/mL) |
| 30 mg/mL | Water | None | 0.000 | 31.8 |
| siRNA in | | | | |
| Milli-Q | | | | |
| Water | | | | |
| 30 mg/mL | 0.5x | 69 | 85.1 | 111.4 |
| siRNA in | PBS | | | |
| Milli-Q | | | | |
| Water | | | | |
| 30 mg/mL | 1x | 138 | 170.2 | 148.4 |
| siRNA in | PBS | | | |
| Milli-Q | | | | |
| Water | | | | |

As shown in Table 2, increasing DF solution salt concentration was associated with increased retentate siRNA concentration. A concentration of about 150 mg/ml of the siRNA molecule in the retentate was achieved using 1×PBS as the DF solution. The % recovery of the siRNA molecule in the final retentate was >85%. Also, flux was maintained for a longer duration when 1×PBS was used as the DF solution (relative to the other DF solutions). A sudden increase in flux was observed when the DF solution was water. The sudden change in flux was indicative of a rupture of the membrane caused by high membrane pressure/fouling. These results demonstrated that higher DF solution salt concentration associates with a higher siRNA concentration in the retentate and a longer time during which membrane flux is maintained.

### Example 3

This example describes the evaluation of a PES membrane for use in UF/DF.

UF/DF was carried out as essentially described in Example 2 except that the membrane used was a 5-kDa Sartorius Polyethersulfane (PES) membrane. The starting solution comprised the siRNA molecule (30 mg/mL) in water, and the DF solution comprised a 1×PBS. Under these conditions, the siRNA concentration of the retentate increased 5-fold from 30 mg/mL to 151.7 mg/mL. The % recovery of the siRNA molecule in the final retentate was >85%. These results suggest that a PES membrane, like the 5 kDa MWCO stabilized cellulose, is also suitable for UF/DF of an siRNA molecule.

### Example 4

This example describes the effect of high salt concentrations in the DF solutions.

To further evaluate a correlation between DF solution salt concentration and the retentate siRNA concentration, the study of Example 2 was carried out with DF solutions comprising higher salt concentrations. The sodium chloride concentration of the DF solutions tested in this study ranged from 40 mM to 690 mM. The DF solution comprising the highest sodium chloride concentration, 5×PBS, was prepared by dilution of Dulbecco's 10× phosphate buffered saline (dPBS). dPBS comprised the following: 9.0 mM calcium chloride, 4.9 mM magnesium chloride, 26.7 mM potassium chloride, 14.7 mM potassium phosphate monobasic, 1379.3 mM sodium chloride, and 80.6 mM sodium phosphate dibasic. A DF solution comprising an intermediate sodium chloride concentration, 2×PBS, was also made by diluting dPBS. The DF solution comprising the lowest sodium chloride concentration was a buffer solution comprising 20 mM phosphate buffer and 40 mM sodium chloride.

In this study, a UF/DF system fitted with a 5 kDa MWCO PES membrane was used. The starting solution comprised 50 mg/mL±10 mg/mL siRNA (same molecule as described in Example 1 and FIG. 3) in water. About 5 diafiltration volumes were performed to ensure complete exchange into the DF solution. Process parameters, including transmembrane pressure (TMP), required to facilitate the ultrafiltration process, were maintained consistent across all experiments. Retentate siRNA concentration achieved in each run was measured using a UV spectrometer at 260 nm. The results are summarized in Table 3.

TABLE 3

| | | NaCl | Total salt | |
|---|---|---|---|---|
| | | Concentration | concentration | Retentate |
| | | of the DF | of DF | siRNA |
| Starting | DF | solution | solution | Concentration |
| Solution | Solution | (mM) [symbol] | (mM) | (mg/mL) |
| 50 mg/mL | Buffer | 40 | 84 | 130.4 |
| siRNA in | solution* | | | |
| Milli-Q | | | | |
| Water | | | | |
| 50 mg/mL | 1x PBS | 138 | 170.0 | 149.0 |
| siRNA in | | | | |
| Milli-Q | | | | |
| Water | | | | |
| 50 mg/mL | 2x PBS | 276 | 340.4 | 194.3 |
| siRNA in | | | | |
| Milli-Q | | | | |
| Water | | | | |
| 50 mg/mL | 5x PBS | 690 | 850.0 | 172.9 |
| siRNA in | | | | |
| Milli-Q | | | | |
| Water | | | | |

*20 mM phosphate buffer with 40 mM NaCl

The % recovery of the siRNA duplex in the final retentate for each UF/DF was >80%.

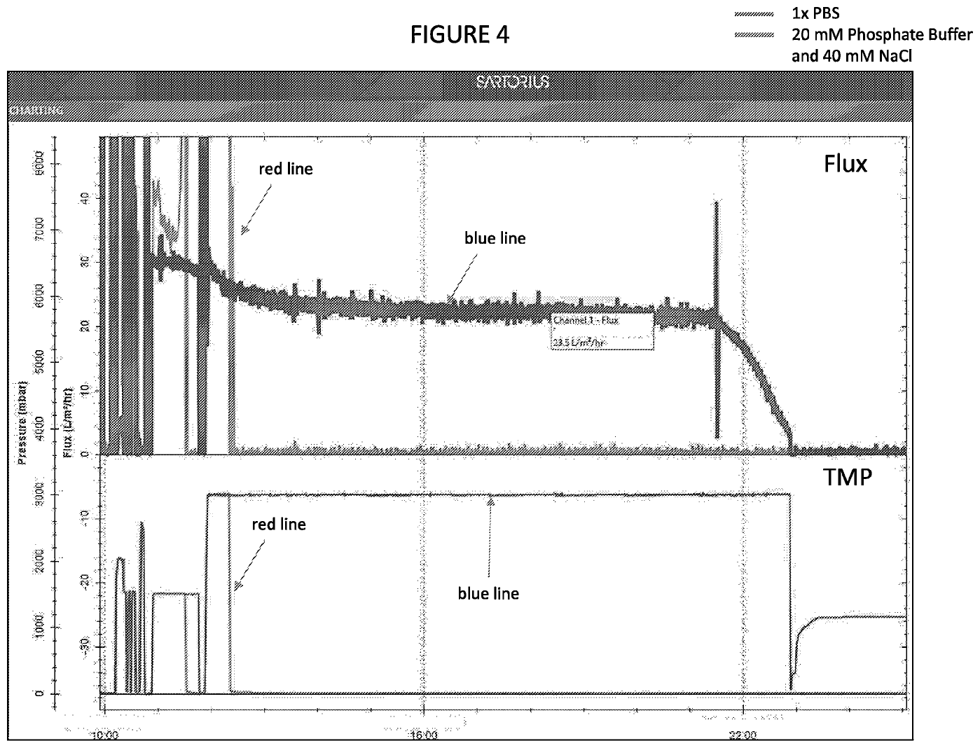

FIG. 4 is a pair of graphs showing the exemplary membrane flux (top) and transmembrane pressure (bottom) as a function of time during UF/DF when the DF solution is buffer solution with 20 mM phosphate buffer and 40 mM sodium chloride (red) or 1×PBS (blue). As shown in FIG. 4, a drastic change in flux and TMP is observed much earlier during the time course when the DF solution has a lower salt concentration (60 mM total salt concentration of formulation buffer—red line) compared to a DF solution having a higher salt concentration (150.2 mM total salt concentration of 1×PBS—blue line). This figure supports that higher DF solution salt concentration associates with a longer time during which membrane flux is maintained.

As shown in Table 3, increasing salt concentration in the DF solution associated with increased retentate siRNA concentration. Each UF/DF carried out using a DF solution comprising a high salt concentration resulted in a retentate siRNA concentration of about 150 mg/mL or greater. Interestingly, the 2×PBS DF solution led to a higher retentate siRNA concentration than that achieved with 5×PBS DF solution (about 194.3 mg/mL compared to 172.9 mg/mL). Without wishing to be bound by any particular theory, it is hypothesized that once the active functional groups of the negatively-charged siRNA molecule are saturated with ionic interactions with cations, further increase in salt concentration does not help to increase the retentate concentration and the DS solution salt concentration effect plateaus. Taken together, these results suggest that, in general, higher salt concentrations of the DF solution led to higher retentate siRNA concentrations and therefore increasing salt concentration in the DF solution is an effective approach for obtaining high concentration solutions of oligonucleotide compounds.

### Example 5

This example describes the evaluation of divalent cationic salts in the DF solution.

In Examples 2 to 4, the salt concentrations of the DF solutions were based on sodium chloride content as this salt was present in dominant amounts relative to the amounts of the other salts present in the DF solutions. To evaluate the impact of the type of salt in the DF solutions on siRNA concentration in the retentate, a DF solution comprising a divalent salt, magnesium chloride, in water was used. The UF/DF conditions were the same as those described in Example 4, except that the DF solution comprised 138 mM magnesium chloride in water. Details of this experiment and the resulting retentate siRNA concentration are provided in Table 4.

TABLE 4

| | | | Retentate |
|---|---|---|---|
| | | MgCl₂ | siRNA |
| Starting | | Concentration | concentration |
| Solution | DF Solution | (mM) | (mg/mL) |
| 40 ± 10 mg/mL | 138 mM magnesium | 138.0 | 209.1 |
| siRNA in water | chloride in water | | |

The % recovery of the siRNA duplex in the final retentate was >85%. As shown in Table 4, use of the DF solution comprising 138 mM of the divalent cationic salt resulted in a higher retentate siRNA concentration (˜209.1 mg/mL). This retentate siRNA concentration was higher than that achieved with the same concentration of the monovalent cationic salt sodium chloride (149.0 mg/mL; see Table 3). Taken together, these results support that divalent salts show a pronounced effect on siRNA concentration during the ultrafiltration process.

### Example 6

This example describes an evaluation of salt concentration in the DF solution on the stability and structure of an siRNA molecule.

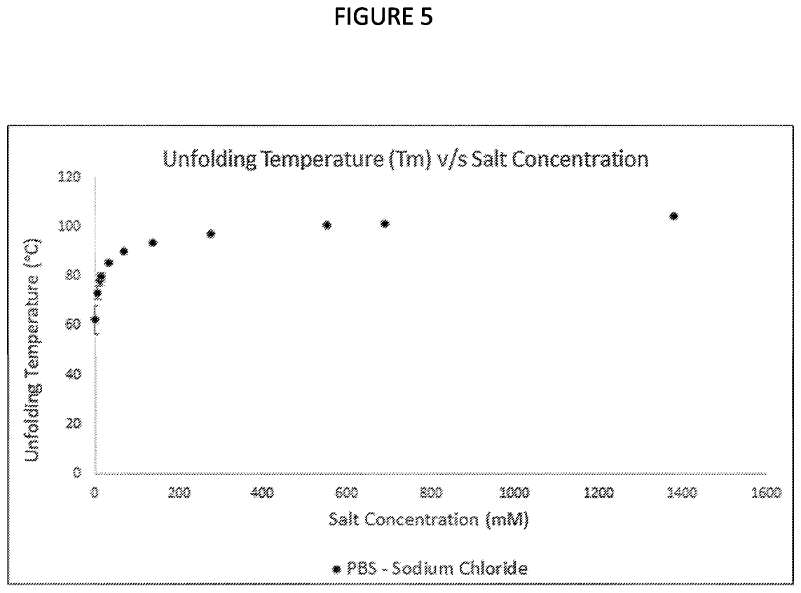

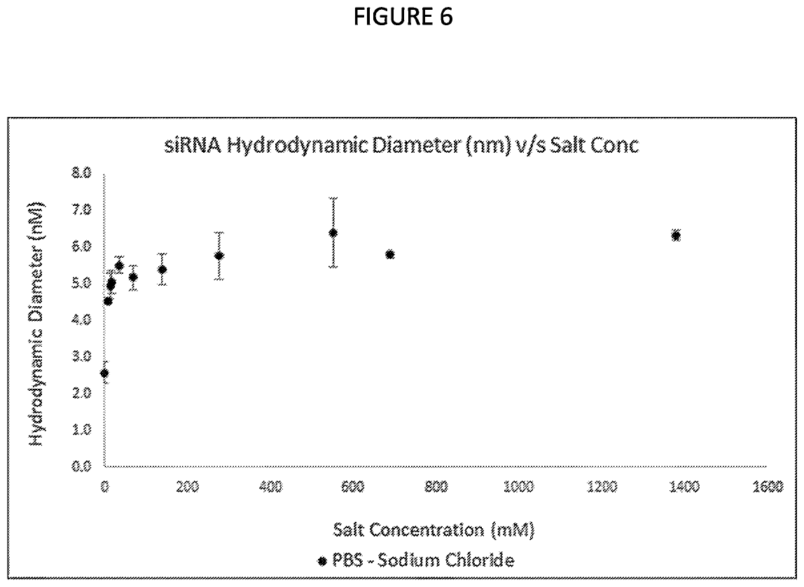

To gain a better understanding of the relationship between increased retentate siRNA concentration and high salt concentration in the DF solution, selected physical properties of the siRNA molecule were evaluated in the presence and absence of salts. The same siRNA molecule described in Example 1 and FIG. 3 was used for these experiments. The siRNA duplex unfolding temperature (Tₘ) and the hydrodynamic diameter (dₕ) were measured using Differential Scanning calorimetry (DSC) and Malvern Mastersizer, respectively. The results are shown in FIG. 5 and FIG. 6. As shown in FIG. 5, the unfolding temperature increases as salt concentration increases. As the unfolding temperature gives an indication of the energy required for denaturing the siRNA duplex, these data suggest that the stability of the siRNA duplex increases as the salt concentration in the DF solution increases.

As shown in FIG. 6, an increasing salt concentration (up to about 600 mM) correlates with increased hydrodynamic diameter, suggesting that the salt interacts with the siRNA molecule in a way that increases the hydrodynamic diameter and leads to a structure which is different from the structure of this molecule in the absence of any salt. Without being bound to a particular theory, it is postulated that the difference in structure of the siRNA duplex in the presence of salt allows for improved flux with the membrane, leading to higher retentate siRNA concentrations.

### Example 7

This example describes an evaluation of various inorganic salt solutions for preparing a high concentration oligonucleotide compound solution.

Results from our earlier studies support that salt solutions comprising a total salt concentration of about 138 mM to about 850 mM can be used as a DF solution in UFDF to obtain retentates comprising high concentration (greater than 150 mg/mL) of the oligonucleotide compound. To better understand the type of salts that may be used for this purpose, solutions comprising different inorganic salts were made and used to concentrate (via ultrafiltration) a low concentration solution of an oligonucleotide compound.

In this study, ultrafiltration and diafiltration were carried out using a high throughput, liquid dispensing instrument fitted with a 3 kDa cellulose membrane. Starting solutions comprising 200 mM inorganic salt and an siRNA at a concentration less than 140 mg/mL were prepared by adding a small volume of an siRNA stock solution to a solution comprising an inorganic salt. The final siRNA concentration and final inorganic salt concentration of each starting solution was 84 mg/mL and 200 mM, respectively. An aliquot of each starting solution was placed into a well of multi-well plate and ultrafiltration was carried out. Each starting solution was pressured-forced through a membrane positioned at the bottom of each well, whereupon water and inorganic salt were collected as permeate in a permeate vessel and the siRNA would remain in the well. The volume of each well of the multi-well plate was monitored by the laser-based detector of the instrument, and when the detected volume was lower than a pre-selected volume, the instrument caused DF solution to be added to the well. The DF solution was identical to the starting solution without siRNA. The process continued until all samples reached a target volume. Run times were typically about 12 hours. The siRNA used in this study was the same siRNA described in Example 1 (hereinafter called “siRNA #1”). Samples were run in triplicate. siRNA concentration after the 12-hour run was measured using a UV spectrometer at 260 nm. A summary of the solutions and the resulting siRNA concentration (reported as an average of three datapoints) is summarized in Table 5. The standard deviation is provided in the right-most column.

TABLE 5

| | | Average | |
|---|---|---|---|
| | | Retentate | |
| | | siRNA #1 | |
| | | Concentration | Standard |
| Starting Solution | DF solution | (mg/mL) | Deviation |
| 84 mg/mL siRNA #1 | 200 mM | 182.1 | 21.2 |
| in 200 mM NH₄Cl | NH₄Cl | | |
| 84 mg/mL siRNA #1 | 200 mM | 220.7 | 32.1 |
| in 200 mM KCl | KCl | | |
| 84 mg/mL siRNA #1 | 200 mM | 186.8 | 2.4 |
| in 200 mM LiCl | LiCl | | |
| 84 mg/mL siRNA #1 | 200 mM | 204.6 | 7.2 |
| in 200 mM NaBr | NaBr | | |

As shown in Table 5, each of the starting solutions comprising 200 mM inorganic salt and 84 mg/mL siRNA was successfully concentrated to an siRNA concentration greater than 150 mg/mL. Starting solutions comprising alkali metal salts KCl and LiCl worked well for this purpose. Starting solutions comprising sodium bromide also achieved high siRNA concentrations suggesting that the inorganic salt can have a counterion other than chloride. Furthermore, the ammonium chloride solution worked well to achieve a high siRNA concentration retentate, supporting that inorganic salts other than alkali metal salts may be used to concentrate the siRNA.

Based on the results presented in Table 5, a theoretical minimum concentration of each salt needed to achieve 150 mg/mL retentate oligonucleotide concentration was calculated. The theoretical minimum concentration for KCl and LiCl was 83.3 mM and 111.7 mM respectively, while the theoretical minimum concentration for ammonium chloride and sodium bromide were 117.3 mM and 94.8 mM, respectively.

### Example 8

This example describes an evaluation of inorganic salts comprising divalent cations for preparing a high concentration oligonucleotide compound solution.

In an earlier study (Example 5), use of a DF solution comprising an inorganic salt comprising a divalent cation at a concentration of 138 mM achieved an oligonucleotide compound concentration of 209 mg/mL. To determine if other inorganic salts comprising a divalent cation could be used to achieve a high concentration oligonucleotide retentate, a solution comprising calcium chloride was prepared. The oligonucleotide compound used in this study was siRNA #1. Because low solubility of the siRNA was observed in 200 mM calcium chloride, the siRNA was prepared in 50 mM calcium chloride. Concentration of a starting solution comprising 84 mg/mL siRNA in 50 mM calcium chloride was carried out as essentially described in Example 7. Samples were run in triplicate. siRNA #1 concentration after the 12-hour run was measured using a UV spectrometer at 260 nm. A summary of the solutions and the resulting siRNA concentration (reported as an average of three datapoints) is summarized in Table 6. The standard deviation is provided in the right-most column.

TABLE 6

| | | Average | |
|---|---|---|---|
| | | Retentate | |
| | | siRNA #1 | |
| | | Concentration | Standard |
| Starting Solution | DF solution | (mg/mL) | Deviation |
| 84 mg/mL siRNA #1 | 50 mM CaCl₂ | 176.84 | 22.5 |
| in 50 mM CaCl₂ | | | |

As shown in Table 6, the starting solution comprising 50 mM calcium chloride and 84 mg/mL siRNA was successfully concentrated to an siRNA concentration greater than 150 mg/mL. Based on the results presented in Table 6, a theoretical minimum concentration of calcium chloride needed to achieve 150 mg/mL siRNA concentration was calculated as 31 mM. These results suggest that the total salt concentration of a DF solution may be as low as 31 mM to arrive at a retentate comprising about 150 mg/mL siRNA.

### Example 9

This example describes an evaluation of organic salt solutions for preparing a high concentration oligonucleotide compound solution.

To determine if organic salts could be used to achieve a high concentration oligonucleotide retentate, a solution comprising choline chloride (CCl), tetramethylammonium chloride (TMACl) and benzyltrimethylammonium chloride (BTMACl) was used to prepare a starting solution comprising a low concentration of siRNA. The siRNA used in this study was siRNA #1. Concentration of a starting solution comprising 84 mg/mL siRNA in 200 mM CCl, TMACl or BTMACl was carried out as essentially described in Example 7. Samples were run in triplicate. siRNA concentration after the 12-hour run was measured using a UV spectrometer at 260 nm. A summary of the solutions and the resulting siRNA concentration (reported as an average of three datapoints) is summarized in Table 7. The standard deviation is provided in the right-most column

TABLE 7

| | | Average | |
|---|---|---|---|
| | | Retentate | |
| | | siRNA #1 | |
| | | Concentration | Standard |
| Starting Solution | DF Solution | (mg/mL) | Deviation |
| 84 mg/mL siRNA #1 | 200 mM | 195.3 | 17.3 |
| in 200 mM CCl | CCl | | |
| 84 mg/mL siRNA #1 | 200 mM | 111.5 | 5.2 |
| in 200 TMACl | TMACl | | |
| 84 mg/mL siRNA #1 | 200 mM | 140.4 | 3.8 |
| in BTMACl | BTMACl | | |

As shown in Table 7, only the CCl solution successfully concentrated the siRNA to greater than 150 mg/mL. Based on the results presented in Table 7, a theoretical minimum concentration of each salt needed to achieve 150 mg/mL retentate oligonucleotide concentration was calculated. The theoretical minimum concentration for CCl, TMACl and BTMACl was 102.9 mM, 460.0 mM and 209.2 mM, suggesting that higher concentrations of BTMACl or TMACl may lead to 150 mg/mL siRNA concentration.

### Example 10

This example describes a study with a different oligonucleotide compound.

Thus far, the experiments described herein have been carried out with siRNA #1. In this study, a different siRNA (siRNA #2) was used. siRNA #2 was a double-stranded siRNA molecule that selectively targets patatin like phospholipase domain containing 3 (PNPLA3). The sense strand of the siRNA #2 is 21 nucleotides long and the antisense strand is 23 nucleotides long. Like siRNA #1, siRNA #2 contains several modified nucleotides and a targeting ligand comprising N-acetylgalactosamine linked to the 5′ end of the sense strand for selective liver targeting.

A starting solution comprising 80 mg/mL siRNA #2 and 200 mM NaCl was prepared, and concentration of this starting solution was carried out as essentially described in Example 7. Samples were run in triplicate. siRNA concentration after the 12-hour run was measured using a UV spectrometer at 260 nm. A summary of the solutions and the resulting siRNA concentration (reported as an average of three datapoints) is summarized in Table 8. The standard deviation is provided in the right-most column.

TABLE 8

| | | Average | |
|---|---|---|---|
| | | Retentate | |
| | | siRNA | |
| | | Concentration | Standard |
| Starting Solution | DF Solution | (mg/mL) | Deviation |
| 80 mg/mL siRNA #2 | 200 mM NaCl | 213.2 | 7.1 |
| in 200 mM NaCl | | | |

As shown in Table 8, the starting solution comprising a low concentration of siRNA #2 was successfully concentrated to greater than 150 mg/mL. Based on the results presented in Table 8, a theoretical minimum concentration of each salt needed to achieve 150 mg/ml retentate oligonucleotide concentration was calculated. The theoretical minimum concentration for NaCl was 88.3 mM. These results suggest that the total salt concentration of a DF solution may be as low as 88 mM to arrive at a retentate comprising about 150 mg/mL siRNA.

### Example 11

This example describes an exemplary method of preparing a high concentration, liquid composition comprising an oligonucleotide compound.

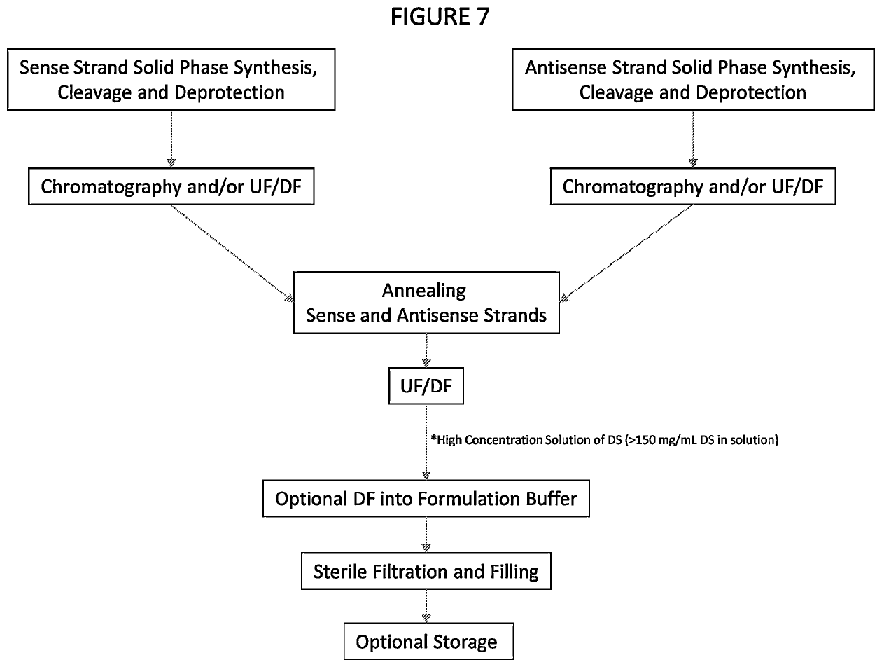

A high concentration, liquid composition comprising a double-stranded (ds) siRNA is made as follows: each of the sense strand and the antisense strand is made via solid phase synthesis. Following cleavage of the synthesized strand from the solid support and deprotection of the heterocyclic bases and backbone, a first UF/DF is carried out to concentrate the strand and exchange it into a new buffer suitable for chromatography. The buffer exchanged strand is then subjected to chromatographic separation to remove impurities. A second UF/DF is carried out to concentrate the strand and exchange it into a new buffer suitable for the strand annealing process. The sense strand and antisense strand are combined for the strand annealing process to obtain ds siRNA. The concentration of the ds siRNA after the strand annealing process is typically in the range of 25 mg/mL to 50 mg/mL. A final UF/DF is carried out to concentrate the double-stranded siRNA to a concentration greater than 150 mg/mL. In this final UF/DF, a diafiltration solution comprising about 50 mM to about 150 mM salt comprising a divalent cation or about 75 mM to about 500 mM salt comprising a monovalent cation is used. The high concentration, liquid composition comprising ds siRNA is either stored frozen or subjected to a series of filtrations for bioburden reduction and sterilization prior to being filled into syringes, autoinjectors, or vials. The filled syringes, autoinjectors, or vials are then stored until shipped. A simplified schematic of the process is provided in FIG. 7.

The above method does not include a lyophilization step and thus provides a less energy intensive- and more cost-efficient way of obtaining a liquid, e.g., aqueous solution, comprising a high concentration of an oligonucleotide compound drug substance.

SEQUENCE TABLE

| SEQ ID NO: | Short Name | Sequence (5′→3′) | Description |
|---|---|---|---|
| 1 | Sense Strand | CAG CCC CUU AUU GUU AUA | |
| | Unmodified | CGA | |
| | Sequence | | |
| 2 | Antisense | UCG UAU AAC AAU AAG GGG | |
| | Strand | CUG | |
| | Unmodified | | |
| | Sequence | | |
| 3 | Sense Strand | CAG CCC CUU AUU GUU AUA | Wherein: |
| | modified | CGA | Each of the nucleotides at positions 1-8 |
| | Sequence | | and 12-20 is a 2′-O-methyl nucleotide |
| | | | Each of the nucleotides at positions |
| | | | 9-11 is a 2′-deoxy-2′-fluoro nucleotide |
| | | | the nucleotide at position 21 is a |
| | | | deoxynucleotide |
| | | | the nucleotides at positions 1 and 2 are |
| | | | linked by a phosphorothioate bond |
| | | | the nucleotides at positions 20 and 21 |
| | | | are linked by a phosphorothioate bond |
| | | | The nucleotide at position 1 is attached |
| | | | to R1 by a phosphorothioate bond, |
| | | | wherein R1 is an N- |
| | | | acetylgalactosamine glycopeptide |
| | | | The nucleotides at positions 20 and 21 |
| | | | are linked by a 3′-3′ linkage |
| 4 | Antisense | UCG UAU AAC AAU AAG GGG | Wherein: |
| | Strand | CUG | Each of the nucleotides at positions 1, |
| | modified | | 3, 5, 7-11, 13, 15, 17, 19, and 21 is a |
| | Sequence | | 2′-O-methyl nucleotide |
| | | | Each of the nucleotides at positions 2, |
| | | | 4, 6, 12, 14, 16, 18, and 20 is a |
| | | | 2′-deoxy-2′-fluoro nucleotide |
| | | | the nucleotides at positions 1 and 2 are |
| | | | linked by a phosphorothioate bond |
| | | | the nucleotides at positions 2 and 3 are |
| | | | linked by a phosphorothioate bond |
| | | | the nucleotides at positions 19 and 20 |
| | | | are linked by a phosphorothioate bond |
| | | | the nucleotides at positions 20 and 21 |
| | | | are linked by a phosphorothioate bond |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of preparing a high concentration, liquid composition comprising an oligonucleotide compound, said method comprising:
a. exchanging by diafiltration an oligonucleotide compound in a starting solution into a diafiltration (DF) solution to obtain an intermediate solution, wherein the concentration of the oligonucleotide compound in the starting solution is 140 mg/ml or less and the DF solution comprises one or more salts and the total salt concentration of the DF solution is about 25 mM to about 800 mM, and
b. concentrating by ultrafiltration the oligonucleotide compound in the intermediate solution to obtain a high concentration, liquid composition, wherein the concentration of the oligonucleotide compound in the high concentration, liquid composition is greater than about 150 mg/mL.

2. The method of claim 1, wherein the diafiltration is continuous diafiltration and/or the ultrafiltration is ultrafiltration by tangential flow filtration.

3. The method of claim 1 or 2, wherein the method employs a polyethersulfone (PES) membrane or a stabilized cellulose membrane for the diafiltration and/or ultrafiltration.

4. The method of any one of the preceding claims, wherein the membrane has a molecular weight cut-off (MWCO) of less than 10 kDa.

5. The method of claim 4, wherein the membrane has a MWCO of about 5 kDa or less than 5 kDa

6. The method of claim 5, wherein the membrane has a MWCO of about 3 kDa.

7. The method of any one of the preceding claims, wherein the DF solution comprises, consists essentially of, or consists of an inorganic salt.

8. The method of claim 7, wherein the inorganic salt comprises a monovalent cation.

9. The method of claim 7 or 8, wherein the inorganic salt comprises an alkali metal, optionally, sodium, potassium, or lithium.

10. The method of any one of claims 7 to 9, wherein the inorganic salt comprises a halogen counterion, optionally chloride or bromide.

11. The method of any one of claims 7 to 10, wherein the inorganic salt is sodium chloride, sodium bromide, potassium chloride, or lithium chloride.

12. The method of claim 8, wherein the monovalent cation is an ammonium cation, optionally, wherein the inorganic salt is ammonium chloride.

13. The method of claim 7, wherein the inorganic salt comprises a divalent cation.

14. The method of claim 13, wherein the divalent cation is an alkaline earth metal, optionally, magnesium or calcium.

15. The method of claim 14, wherein the inorganic salt is magnesium chloride or calcium chloride.

16. The method of any one of claims 1 to 6, wherein the DF solution comprises, consists essentially of, or consists of an organic salt.

17. The method of claim 16, wherein the organic salt comprises a quaternary ammonium cation, optionally, wherein the organic salt is choline chloride or benzyltrimethylammonium chloride

18. The method of any one of the preceding claims, wherein the total salt concentration of the DF solution is about 25 mM to about 500 mM.

19. The method of claim 18, wherein the total salt concentration of the DF solution is about 25 mM to about 250 mM.

20. The method of claim 19, wherein the total salt concentration of the DF solution is about 25 mM to about 150 mM

21. The method of claim 18, wherein the DF solution comprises, consists essentially of, or consists of an inorganic salt comprising a divalent cation, optionally, calcium chloride or magnesium chloride.

22. The method of claim 21, wherein the total salt concentration of the DF solution is about 75 mM to about 300 mM.

23. The method of claim 18, wherein the total salt concentration of the DF solution is about 75 mM to about 250 mM.

24. The method of claim 22 or 23, wherein the DF solution comprises, consists essentially of, or consists of an inorganic salt comprising a monovalent cation.

25. The method of claim 24, wherein the DF solution comprises about 75 mM to about 300 mM sodium chloride, sodium bromide, lithium chloride, potassium chloride, or ammonium chloride.

26. The method of any one of the preceding claims, wherein the total salt concentration of the DF solution per one negative charge of the oligonucleotide compound is about 3.0 mM to 18.0 mM.

27. The method of claim 26, wherein the total salt concentration of the DF solution per negative charge of the oligonucleotide compound is at least about 3.0 mM or about 3.5 mM.

28. The method of any one of the preceding claims, wherein the DF solution comprises a primary salt and at least one other salt.

29. The method of claim 28, wherein the concentration of the primary salt is at least 2-fold greater than the concentration of the other salt(s) of the DF solution.

30. The method of claim 29, wherein the concentration of the primary salt is at least 3-fold greater than the concentration of the other salt(s) of the DF solution.

31. The method of any one of the preceding claims, wherein the DF solution comprises a buffer.

32. The method of claim 31, wherein the buffer is phosphate buffered saline (PBS).

33. The method of any one of claims 28-32, wherein the concentration of the primary salt in the DF solution is based on the concentration of the oligonucleotide compound present in the starting solution.

34. The method of any one of claims 28 to 33, wherein the molar ratio of the oligonucleotide compound of the starting solution to the primary salt of the DF solution is about 1:2 to about 1:100, about 1:3 to about 1:80, or about 1:5 to about 1:65.

35. The method of any one of claims 28 to 34, wherein the concentration of the primary salt of the DF solution is at least 80% of the total salt concentration of the DF solution.

36. The method of claim 35, wherein the concentration of the primary salt of the DF solution is at least 90% of the total salt concentration of the DF solution.

37. The method of any one of claims 28-36, wherein the primary salt concentration of the DF solution is about 50 mM to about 500 mM.

38. The method of claim 37, wherein the primary salt concentration of the DF solution is about 100 mM to about 600 mM.

39. The method of claim 38, wherein the primary salt concentration of the DF solution is at least 125 mM.

40. The method of claim 39, wherein the primary salt concentration of the DF solution is at least 130 mM.

41. The method of claim 40, wherein the primary salt concentration of the DF solution is at least 140 mM.

42. The method of any one of claims 28-41, wherein the primary salt comprises a monovalent cation.

43. The method of claim 42, wherein the monovalent cation is sodium.

44. The method of claim 43, wherein the primary salt is sodium chloride.

45. The method of any one of claims 28-44, wherein the primary salt comprises a divalent cation.

46. The method of claim 45, wherein the divalent cation is magnesium.

47. The method of claim 46, wherein the primary salt is magnesium chloride.

48. The method of any one of claims 1 to 8, 10, 12-24, and 26-47, wherein the DF solution is substantially free of potassium.

49. The method of any one of the preceding claims, wherein the final retentate comprises at least 80% of the amount of the oligonucleotide compound of the starting solution.

50. The method of claim 49, wherein the final retentate comprises at least 85% of the amount of the oligonucleotide compound of the starting solution.

51. The method of any one of the preceding claims, further comprising finishing or polishing the final retentate, wherein the method, optionally, further comprises collecting the final retentate and filling it into a container with a total oligonucleotide recovery of greater than 80%.

52. The method of any one of the preceding claims, wherein the method does not comprise lyophilization of the oligonucleotide compound.

53. The method of any one of the preceding claims, wherein the oligonucleotide compound is double stranded or single stranded.

54. The method of any one of the preceding claims, wherein the oligonucleotide compound is an siRNA.

55. The method of any one of the preceding claims, wherein the oligonucleotide compound is an antisense oligonucleotide (ASO).

56. The method of claim 53, wherein the double stranded oligonucleotide compound comprises an antisense oligonucleotide and a sense oligonucleotide, wherein each of the antisense and sense oligonucleotides independently comprises at least 11 nt, optionally about 20-30 nt or about 20-25 nt.

57. The method of any one of the preceding claims, wherein the oligonucleotide compound has a molecular weight greater than about 7,000 daltons.

58. The method of any one of the preceding claims, wherein the concentration of the oligonucleotide compound of the final retentate in the retentate vessel is at least 2-times higher than the oligonucleotide compound concentration of the starting solution.

59. The method of any one of the preceding claims, wherein the concentration of the oligonucleotide compound of the final retentate in the retentate vessel is at least 3-times higher than the oligonucleotide compound concentration of the starting solution.

60. The method of any one of the preceding claims, wherein the concentration of the oligonucleotide compound of the final retentate in the retentate vessel is at least 4-times higher than the oligonucleotide compound concentration of the starting solution.

61. The method of any one of the preceding claims, wherein the concentration of the oligonucleotide compound of the final retentate in the retentate vessel is at least 5-times higher than the oligonucleotide compound concentration of the starting solution.

62. A high concentration, liquid composition prepared by the method of any one of the preceding claims.

63. A frozen preparation made by storing the liquid composition of claim 62 at a temperature below 0° C., optionally, at −20° C.

64. A method of manufacturing a medicament comprising an oligonucleotide compound, comprising carrying out the method of any one of claims 1-61 to obtain a high concentration, liquid composition comprising at least 150 mg/ml of the oligonucleotide compound, formulating the high concentration, liquid composition with a pharmaceutically-acceptable excipient, and filling the formulated high concentration, liquid composition into a container.

65. A method of treating a subject with a disease, said method comprising administering to the subject a medicament manufactured by the method of claim 64 in an amount effective to treat the disease in the subject, optionally, wherein the medicament is administered by injection or infusion.

66. Use of the high concentration, liquid composition of claim 62 in the manufacture of a medicament for treating a disease in a subject.
